(19) United States
(12) Patent Application Publication
(10) Pub. No.: US 2010/0255556 A1
(43) Pub. Date: Oct. 7, 2010

(54) METHODS AND APPARATUS FOR MANIPULATION OF FLUIDIC SPECIES

(75) Inventors: Thomas Hunt, Portland, OR (US); David Issadore, Cambridge, MA (US); Robert Westervelt, Lexington, MA (US)

Correspondence Address: Harvard University & Medical School;c/o Wolf, Greenfield & Sacks, P.C., 600 Atlantic Avenue, Boston, MA, 02210-2206, US

(73) Assignee: President and Fellows of Harvard College, Cambridge, MA (US)

(21) Appl. No.: 12/602,586

(22) PCT Filed: Jun. 26, 2008
(86) PCT No.: PCT/US08/07941

§ 371 Date: May 24, 2010

Related U.S. Application Data
(60) Provisional application No. 60/947,063, filed on Jun. 29, 2007.

(51) Int. Cl. C12N 13/00; C12M 1/42

(52) U.S. Cl. 435/173.1; 435/283.1

(57) ABSTRACT

The present disclosure relates generally to methods and apparatus for manipulating, detecting, imaging, and/or identifying particles, fluids, or other objects via electromagnetic fields, including methods and apparatus for identifying, sorting, splitting, coalescing, and/or reacting such particles, fluids, or other objects. Certain aspects of the invention are generally directe to methods and devices for producing electric or magnetic fields, e.g., from one or more field-generating components (200) (for example, arranged in an array), to control or manipulate a particle, fluid, or other object. For example, a fluidic droplet may be identified, sorted, separated, split, fused or coalesced, mixed, charged, sensed, determined, etc., using various systems and methods as described herein. In some cases, a particle, a fluidic species (e.g., a droplet), or another object may be contained or constrained by one or more layers of fluid. Other aspects of the invention are directed to methods of making such devices, methods of promoting the making or use of such devices, or the like.

### RELATED APPLICATIONS

This application claims the benefit of U.S. Provisional Patent Application Ser. No. 60/947,063, filed Jun. 29, 2007, entitled “Methods and Apparatus for Manipulation of Fluidic Species,” by Hunt, et al., incorporated herein by reference.

### GOVERNMENT FUNDING

Research leading to various aspects of the present invention was sponsored, at least in part, by the National Science Foundation, Grant No. PHY-0117795. The U.S. Government has certain rights in the invention.

### FIELD OF INVENTION

The present disclosure relates generally to methods and apparatus for manipulating, detecting, imaging, and/or identifying particles, fluids, or other objects via electromagnetic fields, including methods and apparatus for identifying, sorting, splitting, coalescing, and/or reacting such particles, fluids, or other objects.

### BACKGROUND

In biological and medical sciences, it is often useful to be able to manipulate (e.g., move or direct) a biological sample (e.g., one or more cells) along a prescribed path. Manipulation of biological systems based on magnetic fields is one conventionally used method to accomplish this task. In one conventional implementation involving magnetic fields, a small magnetic bead with a chemically modified surface can be coupled to a target biological system, such as a particular cell or microorganism. Depending on the type of coating of a given bead, and the relative sizes of the bead and the target cell or microorganism, the bead may be bound to the surface of the cell or organism (exterior coupling), or ingested by the cell or organism (interior coupling). Such a “bead-bound” sample then may be suspended in a host liquid to constitute a “microfluid,” and the suspended sample in the microfluid can then be manipulated using an external magnetic field. Devices based on this principle often are referred to as “magnetic tweezers” and have been conventionally used, for example, to trap small particles (e.g., DNA) suspended in a liquid for study. Because magnetic fields and the magnetic beads themselves are typically biocompatible, this process is non-invasive and generally not damaging to the sample. However, conventional magnetic tweezers fail to provide individual control of multiple magnetic beads because these devices typically produce only a single field peak that may be moved; thus only a single bead or, simultaneously, a group of beads in close proximity, may be conventionally controlled within a microfluid.

Another area related to the movement and manipulation of biological samples, particles, or other objects suspended in liquid involves a phenomenon referred to as “dielectrophoresis.” Dielectrophoresis occurs when an inhomogeneous electric field induces a dipole on a particle suspended in liquid. The subsequent force on the dipole pulls the particle to either a minimum or a maximum of the electric field. Almost any particle, without any special preparation, can be trapped or moved using dielectrophoresis when it is exposed to the proper local electric field. This is an advantage of electric field-based operation over the magnetic field-based manipulation described above, as the latter mandates marking biosamples or other objects of interest with magnetic beads. However, a potential disadvantage of the dielectrophoresis is that a relatively strong electric field may damage the cell, particle, or other object of interest in some circumstances.

Yet another area related to the movement and manipulation of biological samples that enables various applications in medical diagnostics and life sciences is referred to as “microfluidics.” Microfluidics is directed to the containment and/or flow of small biological samples by providing a microscale biocompatible environment that supports and maintains physiological homeostasis for cells and tissues. Microfluidic systems may be configured as relatively simple chambers or reservoirs (“bathtubs”) for holding liquids containing cells or other biological samples of interest; alternatively, such systems may have more complex arrangements including multiple conduits or channels in which cells, particles, or other objects of interest may flow. By controlling the flow of fluids in the microscale channels, a small quantity of samples can be guided in desired pathways within a microfluidic system. Integration of various microfluidic devices, such as valves, filters, mixers, and dispensers, with microfluidic channels in a more complex microfluidic system, facilitates sophisticated biological analysis on the microscale. Fabrication of even some complex conventional microfluidic systems generally is considered to be cost-effective, owing to soft-lithography techniques that allow many replications for batch fabrication.

Once fabricated, however, conventional microfluidic systems (especially more complex systems) do not offer an appreciable degree of flexibility, and specifically suffer from insufficient programmability and controllability. In particular, conventional microfluidic systems that are used for analytic operations such as cell sorting are manufactured to have a specific number and arrangement of fixed channels and valves. Operation of the valves controls the flow of cells into the channels, thereby sorting them. The function of the system generally is based on a statistical approach of differentiating amongst relatively larger numbers of cells, and not sorting one cell at a time. Because the arrangement of channels and valves is determined during fabrication of the microfluidic system, each system is designed for a specific operation and typically cannot be used in a different process without modifying its basic structure.

Integrated circuit (“IC”) technology is one of the most significant enabling technologies of the last century. IC technology is based on the use of a variety of semiconductor materials (e.g., silicon (Si), silicon germanium (SiGe), gallium arsenide (GaAs), indium phosphide (InP), etc.) to implement a wide variety of electronic components and circuits. Perhaps one of the most prevalent examples of IC technology is “CMOS” (Complimentary-Metal-Oxide-Semiconductor) technology, with which silicon integrated circuits are fabricated. CMOS technology is what made possible advanced computation and communication applications that are now a routine part of everyday life, such as personal computers, cellular telephones, and wireless networks, to name a few. The growth of the computer and communication industry has significantly relied upon continuing advances in the electronic and related arts in connection with reduced size and increased speed of silicon integrated circuits, whose trend is often quantified by Moore's law. Currently, silicon CMOS chips can contain over 100 million transistors and operate at multi-gigahertz (GHz) speeds with structures as small as 90 nanometers. CMOS microfabrication technology has matured significantly over the last decades, making silicon integrated circuits very inexpensive. Despite several advantages, however, neither CMOS nor any other semiconductor-based IC technology has been widely used (i.e., beyond routine data processing functions) to implement structures for biological applications such as sample manipulation and characterization.

### SUMMARY OF THE INVENTION

The present disclosure relates generally to methods and apparatus for manipulating, detecting, imaging, and/or identifying particles, fluids, or other objects via electromagnetic fields, including methods and apparatus for identifying, sorting, splitting, coalescing, and/or reacting such particles, fluids, or other objects. The subject matter of the present invention involves, in some cases, interrelated products, alternative solutions to a particular problem, and/or a plurality of different uses of one or more systems and/or articles.

One aspect of the present invention is directed to the manipulation of a sample, such as a fluidic droplet, contained between a fluid separating the sample from a substrate, and a covering fluid. The separating fluid and the covering fluid may be substantially immiscible. The sample may be manipulated using electric and/or magnetic fields, e.g., from one or more field-generating components contained within the substrate. In some cases, the field generating components may be arranged in an array.

Another aspect of the present invention is directed to a method comprising acts of generating one or more electric and/or magnetic fields by activating one or more field-generating components of a plurality of field-generating components contained within a substrate, and manipulating a sample not in direct contact with the substrate using the one or more electric and/or magnetic fields. In another aspect, the invention includes a method of manipulating a fluidic droplet, separated from a substrate by a fluid layer substantially immiscible with the fluidic droplet, in some cases using at least one electric and/or magnetic field generated from an array of field-generating components contained within the substrate.

The present invention, in still another aspect is directed to a method comprising acts of providing a fluidic droplet contained between a first fluid layer and a second fluid layer, wherein the fluidic droplet, the first fluid layer, and the second fluid layer are each substantially immiscible, and manipulating the fluidic droplet using an electric and/or a magnetic field.

The invention, in yet another aspect, is a method that comprises acts of determining a property of a fluidic droplet positioned proximate a substrate containing an array of field-generating components, and manipulating the fluidic droplet using an electric and/or a magnetic field generated by the field-generating components based on this determination.

In one aspect, the invention is directed to a method of generating an electric field having a field strength of less than about 100 kV/m by activating one or more field-generating components of a plurality of field-generating components contained within a substrate, and manipulating a sample not in direct contact with the substrate using the electric field. In another aspect, the invention is directed to a method of generating a magnetic field having a field strength of less than about 100 mT by activating one or more field-generating components of a plurality of field-generating components contained within a substrate, and manipulating a sample not in direct contact with the substrate using the magnetic field. In still another aspect, the invention is a method of manipulating a fluidic droplet using an electric and/or a magnetic field having a field strength imparting a net force per unit volume on the fluidic droplet of no more than about 0.2 pN/micrometer³.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates an exemplary physical arrangement of components of a system according to one embodiment of the present disclosure;

FIGS. 2A-2C illustrate a fluidic droplet proximate a substrate, in certain embodiments of the invention;

FIG. 3 conceptually illustrates two neighboring microcoils of an array used to generate essentially equal magnetic field peaks, according to one embodiment of the present disclosure;

FIGS. 4A-4E show five exemplary scenarios for the neighboring microcoils of FIG. 3, with varying current magnitudes and directions in the respective coils and the resulting magnetic fields generated, according to one embodiment of the present disclosure;

FIG. 5 illustrates the movement of a fluidic droplet relative to a substrate, according to one embodiment of the invention;

FIGS. 6A-6B illustrate the splitting of a fluidic droplet into two fluidic droplets, according to another embodiment of the invention;

FIGS. 7A-7C illustrate the coalescing of two fluidic droplets, according to yet another embodiment of the invention;

FIG. 8 illustrates mixing within a fluidic droplet, in still another embodiment of the invention;

FIGS. 9A-9B illustrate a manipulator chip prepared according to one embodiment of the invention;

FIG. 10 illustrates a circuit diagram of a pixel in a manipulator chip in one embodiment of the invention;

FIG. 11 illustrates a bit line control circuit block diagram, in another embodiment of the invention;

FIG. 12 illustrates a schematic of a control cell, in yet another embodiment of the invention

FIGS. 13A-13C show finite element simulations of another embodiment of the invention;

FIG. 14 is a schematic diagram illustrating a microfluidic system on a chip, in one embodiment of the invention;

FIG. 15 is a photograph of another microfluidic system on a chip, in another embodiment of the invention;

FIGS. 16A-16C illustrate the manipulation of yeast cells according to one embodiment of the invention;

FIG. 17 illustrates the formation of complex patterns using cells manipulated using another embodiment of the invention;

FIGS. 18A-18C illustrate the manipulation of mammalian cells according to another embodiment of the invention;

FIGS. 19A-19H illustrate the splitting, moving, and combination of water droplets in oil, according to still another embodiment of the invention;

FIGS. 20A-20B illustrate the crossing of two fluidic streams of droplets, in yet another embodiment of the invention; and

FIG. 21 illustrates another embodiment of the invention.

### DETAILED DESCRIPTION

The present disclosure relates generally to methods and apparatus for manipulating, detecting, imaging, and/or identifying particles, fluids, or other objects via electromagnetic fields, including methods and apparatus for identifying, sorting, splitting, coalescing, and/or reacting such particles, fluids, or other objects. Certain aspects of the invention are generally directed to methods and devices for producing electric or magnetic fields, e.g., from one or more field-generating components (for example, arranged in an array), to control or manipulate a particle, fluid, or other object. For example, a fluidic droplet may be identified, sorted, separated, split, fused or coalesced, mixed, charged, sensed, determined, etc., using various systems and methods as described herein. In some cases, a particle, a fluidic species (e.g., a droplet), or another object may be contained or constrained by one or more layers of fluid. Other aspects of the invention are directed to methods of making such devices, methods of promoting the making or use of such devices, or the like.

One aspect of the present invention includes a device able to generate one or more electric and/or magnetic fields using one or more electric and/or magnetic field-generating components, for example, contained within a substrate. The electric and/or magnetic field-generating components may be disposed in a variety of arrangements so as to facilitate interactions between generated fields and a sample (for example, a fluidic droplet) that is in proximity with the field-generating components. For instance, the plurality of field-generating components may be present as an array, such as a rectangular or a triangular array. In various implementations, the field-generating components may be arranged so as to permit field-sample interactions. In some cases, as discussed below, the sample is not in direct contact with the field-generating components. One non-limiting example of a system comprising a plurality of electric and/or magnetic field-generating components arranged to be able to interact and/or manipulate a sample is disclosed in U.S. patent application Ser. No. 11/105,322, filed Apr. 13, 2005, entitled “Methods and Apparatus for Manipulation and/or Detection of Biological Samples and Other Objects,” by Ham, et al., published as U.S. Patent Application Publication No. 2006/0020371 on Jan. 26, 2006, incorporated herein by reference. However, as is discussed herein, other systems and system arrangements are also possible.

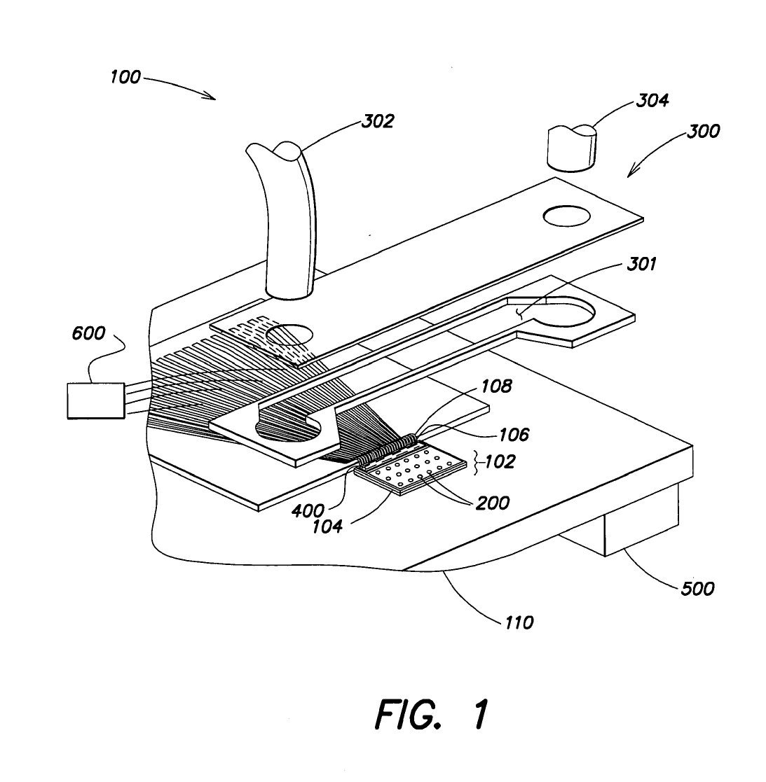

A non-limiting example of a device able to generate one or more electric and/or magnetic fields using field-generating components is illustrated in FIG. 1. This figure illustrates system 100, in which one or more field-generating components 200 may be fabricated on a semiconductor substrate 104, pursuant to any of a variety of semiconductor fabrication techniques, to form IC chip 102. Some or all of these other components of system 100 may be implemented as one or more integrated circuit (IC) chips 102 using various semiconductor fabrication techniques known to those of ordinary skill in the art. For instance, one example implementation of such an IC chip may be fabricated using standard CMOS protocols. It should be appreciated, however, that the present disclosure is not intended to be limiting in this respect, as other semiconductor-based technologies may be utilized to implement various embodiments of the microelectronics portion of the systems discussed herein. IC chip 102, in this example, may be mounted on package substrate 110, and bonding wires 106 and contacts (e.g., pins) 108 may be employed to facilitate electrical connections to the IC chip 102. Other electronic components may be added as well, in various embodiments of the invention. For example, optionally, IC chip 102 may include other components, such as field control components 400 and/or temperature components 500. As another example, IC chip 102 may include various components to facilitate wireless communication of data and control signals to and from IC chip 102. As yet another example, system 100 may include one or more processors 600 configured to control the various components of system 100 to facilitate manipulation of samples such as fluidic droplets, e.g., as described herein. Processors 600 also may be configured to perform various signal processing functions to facilitate detection, imaging, identification, manipulation, etc. of samples. It should be appreciated that in various configurations, processors 600 may be implemented as separate components from the system 100, and optionally located remotely from system 100, as shown in FIG. 1 (e.g., a variety of conventional computing apparatus may be coupled to system 100 via one or more contacts 108, or via wireless communications, etc.). In other instances, however, some or all of the processor functionality may be implemented by elements integrated together with other components in one or more chips 102 that form part of system 100.

Field-generating components 200 may be configured to generate electric fields, magnetic fields, or both. For example, in one embodiment, the field-generating components are configured and operated to produce controllable spatially and/or temporally variable magnetic fields that extend into the microfluidic system. Non-limiting examples of magnetic field-generating components 200 that may be included in system 100 include, but are not limited to, a two-dimensional microelectromagnet wire matrix, as well as one or more “ring traps.” These exemplary components are discussed in detail in, e.g., International Patent Application No. PCT/US02/36280, filed Nov. 5, 2002, entitled “System and Method for Capturing and Positioning Particles,” by Westervelt, et al., published as WO 03/039753 on May 15, 2003, incorporated herein by reference.

Yet other examples of magnetic field-generating components include microscale magnets configured as coils, or “microcoils.” Some examples of microcoils including ferromagnetic cores and fabricated using micromachining techniques are given in U.S. Pat. Nos. 6,355,491 and 6,716,642, as well as International Application Publication No. WO 00/54882, each of which publications is incorporated herein by reference. Yet another example of magnetic field-generating components according to one embodiment of the present invention includes a CMOS microcoil array and associated control circuitry. In some embodiments, the microcoils may include at least two axially concentric spatially separated portions (e.g., layers) of conductor turns. Additional examples of devices including magnetic field-generating components are disclosed in U.S. patent application Ser. No. 11/105,322, filed Apr. 13, 2005, entitled “Methods and Apparatus for Manipulation and/or Detection of Biological Samples and Other Objects,” by Ham, et al., published as U.S. Patent Application Publication No. 2006/0020371 on Jan. 26, 2006, incorporated herein by reference.

The magnetic fields thusly generated can interact with magnetic samples contained inside the fluidic system, examples of which include, but are not limited to, biological cells attached to magnetic beads (“bead-bound cells”), or fluidic droplets such as those discussed below. With respect to biological samples, it is noteworthy that the magnetic fields do not damage cells; rather, as discussed above, cell manipulation and identification via magnetic fields is a commonly used technique to molecularly identify a biological cell by a specific, ligand-coated magnetic bead. As discussed in further detail below, the interaction between the spatially and/or temporally variable magnetic fields and bead-bound cells or other magnetic samples enables trapping, transport, detection, imaging, or manipulation of single or multiple samples.

In some embodiments of the invention, the microcoils can be used to manipulate magnetic beads, e.g., including cells. In some cases, the microcoils can be used to generate a magnetic field able to polarize the magnetic beads. For instance, the amount of energy needed to trap or otherwise manipulate a magnetic bead in such a system may be proportional to the square of the strength of the magnetic field created by the microcoils. In some embodiments, the magnitude of this energy available for trapping or manipulating a magnetic bead can be increased by applying an external magnetic field. For instance, the device may be positioned proximate a permanent magnet or an electromagnet to create the applied external magnetic field. In some cases, the external magnetic field may be one or more orders of magnitude larger than the strength of the magnetic field created by the microcoils. Without wishing to be bound by any theory, it is believed that the application of such a field induces a fixed magnetic polarization inside the magnetic bead that is larger than the polarization created by the magnetic field created by the microcoils. In this situation, the energy for trapping or manipulating a magnetic bead can be orders of magnitude larger than the energy without an external field. Accordingly, in certain embodiments of the invention, an applied external magnetic field is applied to magnetic samples or other species that are manipulated by the microcoils.

In another embodiment, the field-generating components may include an array of microelectrodes, or “microposts,” configured to generate controllable electric fields for manipulating objects of interest, e.g., according to principles of dielectrophoresis. Dielectrophoresis occurs when an inhomogeneous electric field induces a dipole on a material (such as a particle) that is suspended in liquid. The subsequent force on the dipole pulls the particle to either a minimum or a maximum of the electric field. Almost any particle, without any special preparation, can be trapped or moved using dielectrophoresis when it is exposed to the proper local electric field. In this manner, according to one embodiment, one or more samples of interest may be manipulated via operation of a micropost array to generate electric fields appropriate for this task.

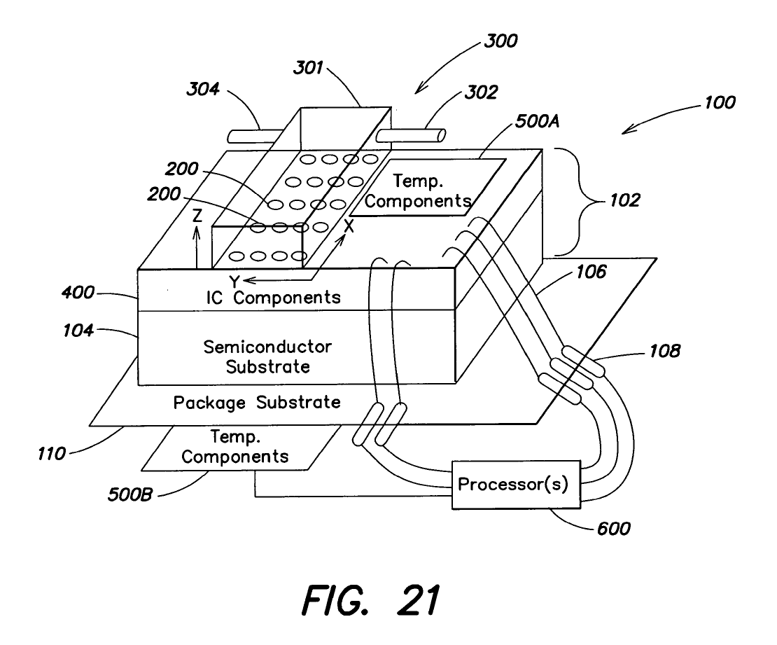

Of course, the invention is not limited to only the configuration described above. Other configurations are also possible, e.g., as is shown in FIG. 21. As another example, it should be appreciated that for virtually any system according to the present disclosure based on a microelectronics portion configured to generate controllable spatially and/or temporally variable magnetic fields, a parallel implementation may be realized using configurations for generating controllable spatially and/or temporally variable electric fields, or a combination of variable magnetic fields and variable electric fields. For example, in yet another embodiment, an array of microcoils may be configured to produce both controllable, spatially and/or temporally patterned, electric fields and/or magnetic fields. For instance, respective independently controllable voltages may be applied across the microcoils of a microcoil array, such that the individual microcoil structures behave essentially like the microposts of a micropost array, namely, by generating electric fields that are capable of interacting with samples contained in the microfluidic system. As a particular example, respective independently controllable currents also may be applied to the microcoils of the microcoil array, to additionally generate magnetic fields that are capable of interacting with magnetic samples contained in the microfluidic system. These and other types of electric field-based or electric/magnetic field-based implementations may be employed for a variety of applications relating to manipulation, sensing and imaging systems that integrate microelectronics and microfluidics.

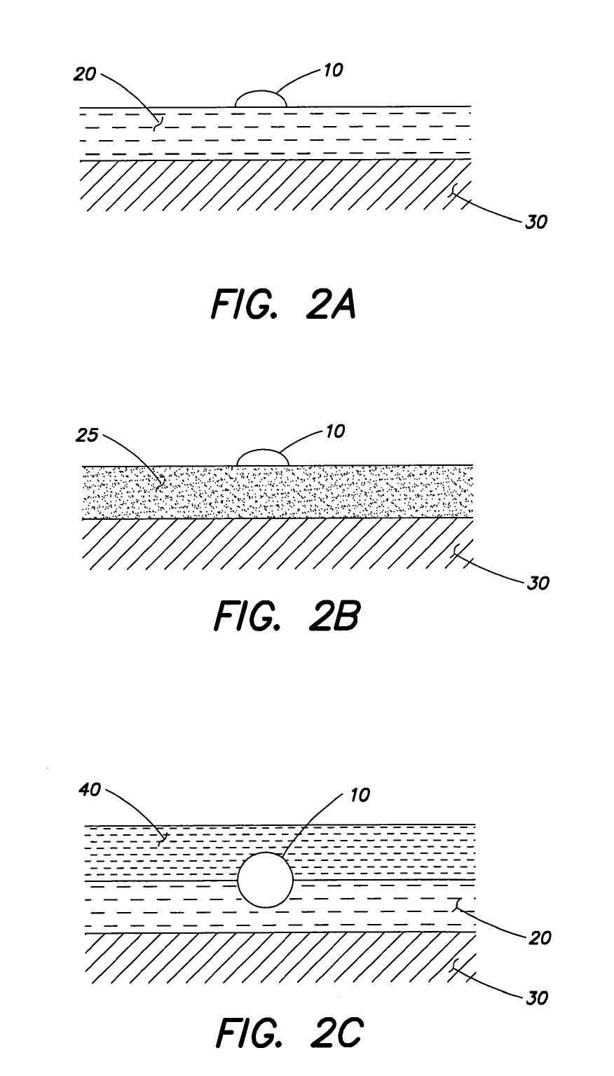

Also shown in FIG. 2 is fluidic system 300, which may be a microfluidic system in some cases, as discussed below. The fluidic system can be positioned such that a sample, such as a fluidic droplet, positioned within the fluidic system can be manipulated using one or more electric and/or magnetic fields generated by one or more of the field-generating components. For example, the field-generating components may be positioned proximate to the fluidic system along one or more physical boundaries of the fluidic system and arranged so as to permit field-sample interactions along one or more spatial dimensions relative to the fluidic system. The sample need not be positioned in direct contact with the field-generating components, but may be positioned proximate to the field-generating components, i.e., positioned such that the fluidic system can be manipulated using one or more electric and/or magnetic fields generated by one or more of the field-generating components. Thus, the electric and/or magnetic field-generating components of the system may be disposed with respect to the microfluidic system in a variety of arrangements so as to facilitate interactions between generated fields and samples contained in (or flowing through) the fluidic system.

The fluidic system may include a relatively simple chamber or reservoir for holding liquids containing samples of interest. For example, as illustrated generically in FIG. 1, a fluidic system can include a chamber 301 having an essentially rectangular shape (or other shape), and channels 302 and 304 to facilitate fluid flow into and out of the chamber. The chamber may have any number of inlets and/or any number of outlets. As shown in the example of FIG. 1, the chamber covers substantially all of the plurality of field-generating components contained within the substrate. Alternatively, the fluidic system may have a more complex arrangement including one or more conduits or channels in which liquids containing samples may flow, as well as various components (e.g., valves, mixers, etc.) for directing flow. In various embodiments, the fluidic system may be fabricated on top of an IC chip containing other system components (e.g., after the semiconductor fabrication processes are completed); alternatively, the fluidic system may be fabricated separately (e.g., using soft lithography techniques) and subsequently attached to one or more IC chips containing other system components. Other examples of suitable fabrication techniques are discussed below.

In some aspects of the invention, the fluidic system may contain a sample containing one or more fluidic droplets, which can be manipulated using electric and/or magnetic fields generated by the field-generating components. The fluidic droplet may be microfluidic in some cases, i.e., having a characteristic dimension of less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases, where the characteristic dimension is the diameter of a perfect sphere having the same volume as the fluidic droplet. The characteristic dimension may also be at least about 1 micrometer, at least about 2 micrometers, at least about 3 micrometers, at least about 5 micrometers, at least about 10 micrometers, at least about 15 micrometers, or at least about 20 micrometers in certain cases. Those of ordinary skill in the art will be able to determine the characteristic dimension, for example, using laser light scattering, microscopic examination, or other known techniques. It is to be noted that the fluidic droplet may not necessarily be spherical, but may assume other shapes as well, for example, depending on the external environment (e.g., by the shape of the conduits containing the fluidic droplet, by fluids flowing in such conduits, by influences due to electric and/or magnetic fields (e.g., if the fluidic droplet is electrically and/or magnetically susceptible), or the like. The fluidic droplet(s) may be surrounded by one or more liquids (e.g., suspended), in some cases, e.g., as discussed below. If more than one fluidic droplet is present within the system, the droplets may be of substantially the same shape and/or size (e.g., the droplets may be monodisperse), or of different shapes and/or sizes, depending on the particular application. The fluidic droplet(s) may also contain other species, for example, certain molecular species (e.g., as further discussed below), cells, particles, etc..

As used herein, the term “fluid” generally refers to a substance that tends to flow and to conform to the outline of its container, i.e., a liquid, a gas, a viscoelastic fluid, etc. Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids.

The fluidic droplets may be formed using any suitable technique, and may be formed within the system or formed externally and transported into the system or into a chamber or reservoir, e.g., via a microfluidic conduit. For example, the droplets may be formed by shaking or stirring a liquid to form individual droplets, creating a suspension or an emulsion containing individual droplets, or forming the droplets through pipetting techniques, needles, or the like. Additional, non-limiting examples of the production and manipulation of droplets of fluid are described in International Patent Application Serial No. PCT/US2004/010903, filed Apr. 9, 2004 by Link, et al., published as WO 2004/091763 on Oct. 28, 2004; International Patent Application Serial No. PCT/US03/20542, filed Jun. 30, 2003 by Stone, et al., published as WO 2004/002627 on Jan. 8, 2004; or U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007, each incorporated herein by reference.

The fluidic droplets may be contained by one or more fluids, according to another aspect of the invention. For example, a fluidic droplet may be surrounded by a liquid. The fluidic droplet and the liquid may be substantially immiscible in many cases, i.e., immiscible on a time scale of interest (e.g., the time it takes a fluidic droplet to be transported through the system, analyzed, etc.). For example, two fluids can be selected to be substantially immiscible within the time frame of formation of a stream of fluids, or within the time frame of reaction or interaction. In some cases, two fluids are substantially immiscible, or not miscible, with each other when one is not soluble in the other to a level of at least 10% by weight. As an example, a hydrophobic liquid and a hydrophilic liquid are substantially immiscible with respect to each other, where the hydrophilic liquid has a greater affinity to water than does the hydrophobic liquid. Examples of hydrophilic liquids include, but are not limited to, water and other aqueous solutions comprising water, such as cell or biological media, salt solutions, etc., as well as other hydrophilic liquids such as ethanol. A hydrophilic liquid, in some cases, can be identified by mixing the hydrophilic liquid with water and determining if phase separation of the hydrophilic liquid and water occurs over an extended time period, e.g., days to weeks. Examples of hydrophobic liquids include, but are not limited to, oils such as hydrocarbons, silicone oils, mineral oils, fluorocarbon oils, organic solvents etc.

In one set of embodiments, a fluidic droplet (or other sample) is separated from a substrate containing one or more electric and/or magnetic field-generating components by a second, separating fluid. More than one separating fluid (or other separating material, as discussed below) may be used in some cases. Referring now to FIG. 2A as an example, fluidic droplet 10 is separated from substrate 30 via separating fluid 20. In some cases, the fluidic droplet and the separating fluid are substantially immiscible. By using a separating fluid (or other separating material), fluidic droplet is prevented from contacting substrate 30, which may be useful to prevent or reduce reaction with the substrate (for example, if fluidic droplet contains a cell or a biological species of interest), and/or to reduce the amount of energy necessary to move the fluidic droplet with respect to the substrate. For instance, the amount of energy needed to move (or otherwise manipulate) the fluidic droplet over the separating fluid may be less than the energy needed to move the fluidic droplet over the substrate if the fluidic droplet was in contact with the substrate. In one embodiment, in the presence of a separating fluid or other separating material, lower energies are needed to move the fluidic droplet with respect to the substrate. For instance, in one embodiment, an electric field having a field strength of less than about 100 kV/m, less than about 50 kV/m, less than about 30 kV/m, less than about 10 kV/m, or less than about 5 kV/m may be sufficient to move or manipulate the fluidic droplet over the separating fluid with respect to the substrate. In another embodiment, a magnetic field having a field strength of less than about 100 mT, less than about 50 mT, less than about 30 mT, less than about 10 mT, or less than about 5 mT may be sufficient to move or manipulate the fluidic droplet over the separating fluid with respect to the substrate. In some cases, an electric and/or a magnetic field having sufficient strength to impart a certain force per unit volume on a droplet is applied. For example, the electric and/or the magnetic field applied to the fluidic droplet may be such that the fluidic droplet feels a net force per unit area of less than about 0.2 pN/micrometer³ (volume of the fluidic droplet), less than about 0.1 pN/micrometer³, less than about 0.05 pN/micrometer³, less than about 0.03 pN/micrometer³, or less than about 0.01 pN/micrometer³. In some cases, such an electric and/or the magnetic field may be able to cause the fluidic droplet to move relative to the substrate, as discussed herein.

In some embodiments of the invention, the fluidic droplet may be separated from the substrate by a material that is not a fluid. For example, as is shown in FIG. 2B, fluidic droplet 10 is separated from substrate 30 by separating material 25. Separating material may be chosen, in some cases, to reduce the amount of energy necessary to move the fluidic droplet with respect to the substrate, and/or to prevent or reduce a reaction of the fluidic droplet (or a species within the fluidic droplet) with the substrate. For example, by using such separating materials, lower electric and/or magnetic field strengths may be necessary to move or manipulate the fluidic droplet. A non-limiting example of such materials is a gel or a hydrogel, for example, agarose, polyacrylamide, gelatin, or the like. Another example is a polymer such as a hydrophobic polymer, for example, polyacrylate, polyacrylonitrile, poly(vinylidene fluoride) and other suitable fluoropolymers, polysulfone, poly(ether sulfone), poly(aryl sulfone), and the like, poly(methyl methacrylate) and polyolefin derivatives, etc., as well as copolymers of these and/or other suitable polymers.

In some embodiments, the fluidic droplet is exposed to the environment, and in some cases, the fluidic droplet may at least partially evaporate (for example, if fluidic droplet contains water or other species having low vapor pressure). In some cases, this effect may be eliminated or at least reduced by using a saturated environment, e.g., saturated in water (saturated relative humidity). However, in another embodiment, the fluidic droplet may be prevented from evaporation by using a covering fluid, or other covering material. More than one such covering fluid and/or material may be used in some cases. Referring now to FIG. 2C as an example, fluidic droplet 10 is contained at the interface between a first, separating fluid 20 and a second, covering fluid 40. The covering fluid may prevent or reduce evaporation of the fluidic droplet. In some cases, the covering fluid is substantially immiscible with the fluidic droplet and/or the separating fluid, and in some cases, the covering fluid has a lower density than the separating fluid. In one embodiment, the covering fluid is transparent or at least substantially transparent.

As mentioned, the fluidic droplet, the separating fluid, and the covering fluid (if present) may each be substantially immiscible in some cases, i.e., immiscible on a time scale of interest. As a specific example, fluidic droplet may be aqueous or hydrophilic (e.g., containing water, biological media, salt solutions, etc., while the separating fluid and the covering fluid are not aqueous or hydrophilic. For instance, the separating fluid may contain a fluorocarbon oil and the covering fluid may contain a hydrocarbon oil, such as hexadecane. Another example of a system involving three substantially mutually immiscible fluids is a silicone oil, a mineral oil, and an aqueous solution (i.e., water, or water containing one or more other species that are dissolved and/or suspended therein, for example, a salt solution, a saline solution, a suspension of water containing particles or cells, or the like). Another example of a system is a silicone oil, a fluorocarbon oil, and water or an aqueous or hydrophilic solution. Yet another example of a system is a hydrocarbon oil (e.g., hexadecane), a fluorocarbon oil, and an aqueous solution. In these examples, any of these fluids may be used as the liquid carrier. Non-limiting examples of suitable fluorocarbon oils include octadecafluorodecahydronaphthalene:

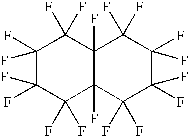

or 1-(1,2,2,3,3,4,4,5,5,6,6-undecafluorocyclohexyl)ethanol:

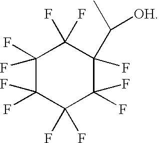

In certain embodiments of the invention, the fluidic droplets may contain additional entities, for example, other chemical, biochemical, or biological entities (e.g., dissolved or suspended in the fluid), cells, particles, gases, molecules, or the like. For instance, the fluidic droplet may contain species such as peptides or proteins, enzymes, antibodies, nucleic acids, polymers, reagents, etc. In some cases, the entities may be sensing entities, for example, which may be fluorescent, luminescent, radioactive, etc. As discussed below, in some cases, the sensing entities may be determined and the information used to manipulate the fluidic droplet.

Various aspects of the present invention are directed to systems and methods of manipulating samples such as fluidic droplets, for example, by moving, splitting, fusing or coalescing, mixing, screening or sorting, sensing or determining, and/or reacting the fluidic droplets and/or species contained within the fluidic droplets. In some cases, one or more electric and/or magnetic field-generating components may be used to manipulate the samples, as is described herein.

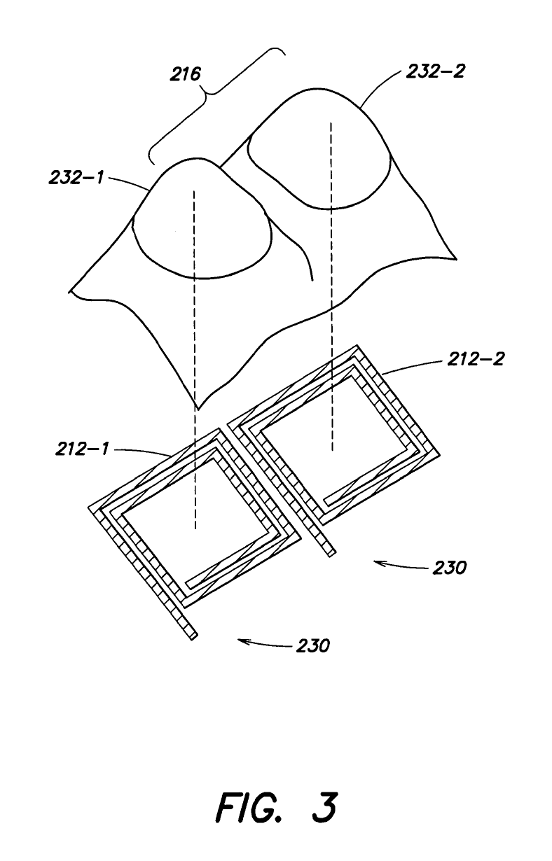

For instance, in one aspect, a fluidic droplet can be moved from a first location to a second location relative to a substrate. By generating electric and/or magnetic fields using one or more electric and/or magnetic field-generating components by activating the components in a specific order, fluidic droplets or other samples can be moved relative to a substrate. As a specific, non-limiting example, if the substrate includes a plurality of microcoils, e.g., arranged in an array, by creating and moving one or more magnetic field peaks by modulating currents in the respective microcoils of the array, samples such as fluidic droplets can be moved via modulation of the magnetic filed peaks, for example, if the fluidic droplet contains a ferrofluid or other magnetizable substance. Ferrofluids are known to those of ordinary skill in the art, and typically contain ferromagnetic particles suspended in a carrier fluid, such as an organic solvent or water, and also often contain a surfactant. The magnitude of the magnetic field generated by a given microcoil of the array is based on the magnitude of the current flowing through the microcoil, and each microcoil in the array is capable of generating a local magnetic field peak above the microcoil. In this sense, the array of microcoils may be thought of generally in terms of “magnetic pixels,” where an N x N array of microcoils is capable of producing at least N×N magnetic peaks, or “pixels,” each capable of attracting and trapping a sample. FIG. 3 conceptually illustrates two neighboring microcoils 212-1 and 212-2 of an array, in which an essentially equal current 230 flows through the microcoils to generate two essentially equal magnetic field peaks 232-1 and 232-2 above the coils. In FIG. 3, the distance between the two magnetic field peaks generally corresponds to the pitch 216 of the array 2008, as indicated in FIG. 3.

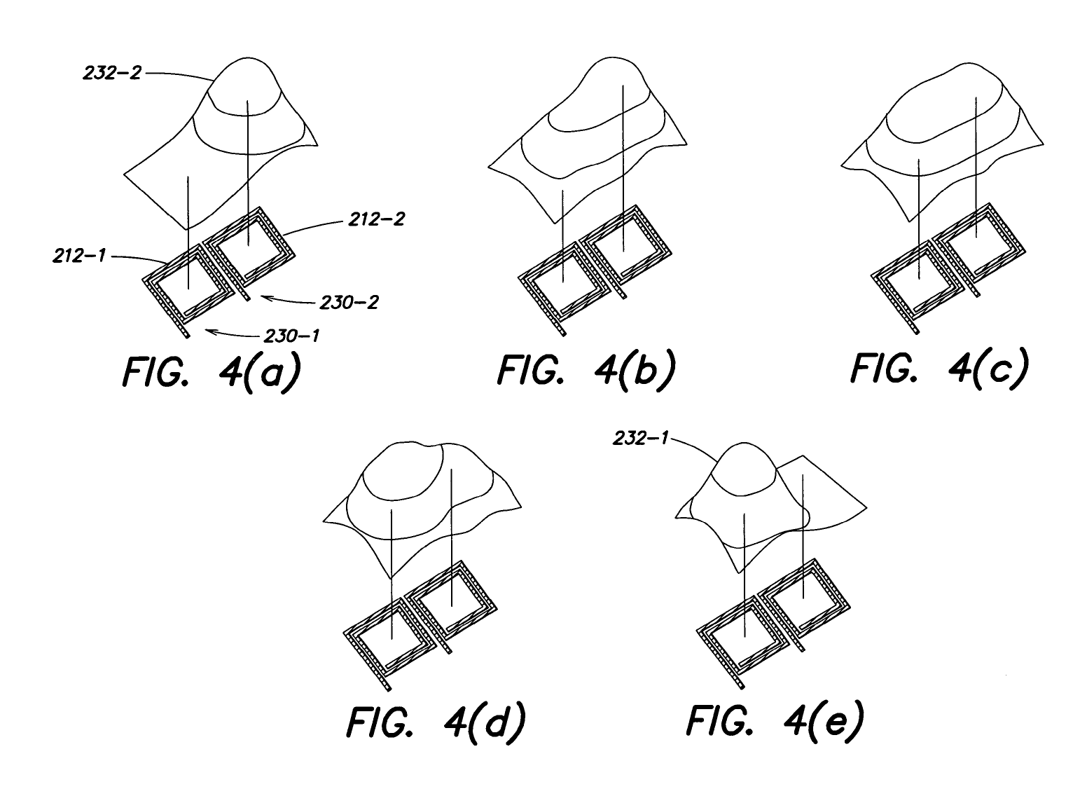

In one embodiment, not only may the magnitude of the current flowing through each microcoil be modulated to facilitate sample manipulation, but also the direction of the current flowing through a given coil may be altered, so as to facilitate a smoother transition of a sample from pixel to pixel, or effectively increase the spatial resolution for sample manipulation (i.e., effectively decrease the pitch 216 of the array). For instance, FIGS. 4A-4E show five exemplary scenarios for the neighboring microcoils 212-1 and 212-2 of FIG. 3, with varying current magnitudes and directions in the respective coils and the resulting magnetic fields generated.

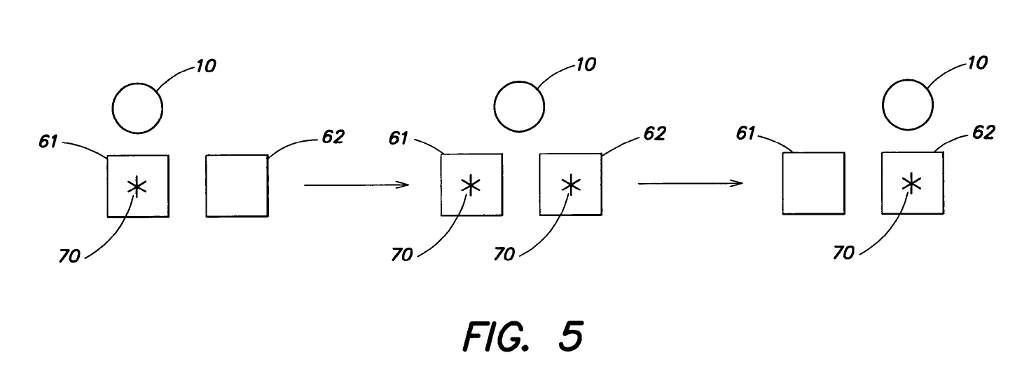

As a more general illustrative example, the process of moving a fluidic droplet from a first location to a second location relative to a substrate is also shown in schematic fashion in FIG. 5, where a first field pixel 61 and a second field pixel 62 are used to move a fluidic droplet 10 from a position above first field pixel 61 to a different position above second field pixel 62. Field pixels 61 and 62 may be defined by field-generating components contained within the substrate. For instance, the field pixels may be able to create magnetic fields (e.g., using microcoils) and/or electric fields. In this figure, the field pixels are activated (e.g., as previously described), as shown by stars 70.

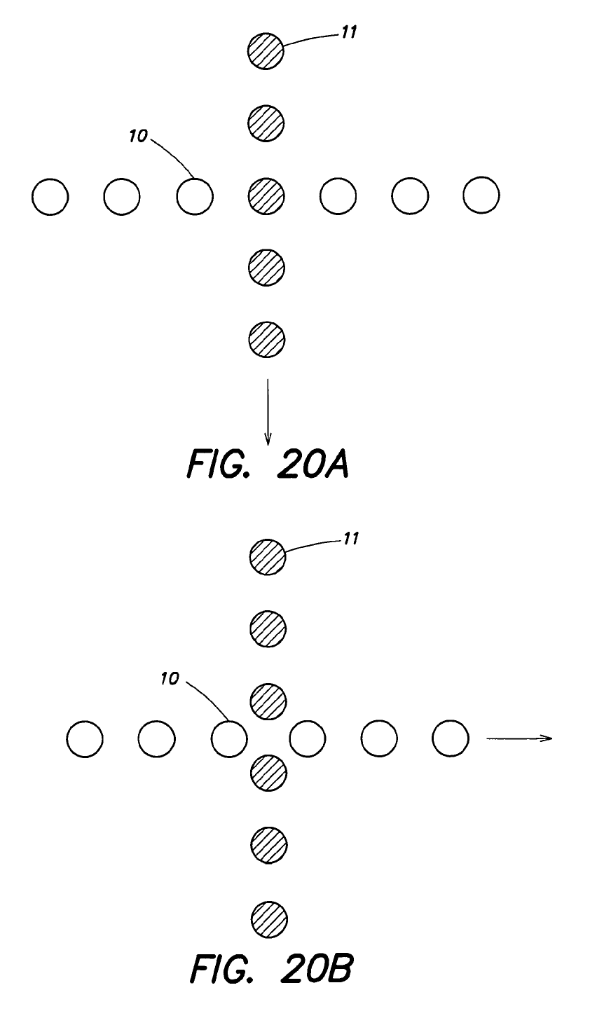

More complex behaviors of droplets (or cells) may be prepared based on techniques such as those described above. For instance, the droplets may be moved in such a fashion in a way that is analogous to moving droplets within fluid channels in. a microfluidic system, but without the need to use actual channels. Thus, the droplets may be moved in parallel, made to stop, made to change direction, etc., without the need to use actual channels to do so. Multiple droplets may be moved indepedently of each other. For instance, the droplets may be moved in parallel, perpendicularly, etc. Two streams of droplets can even be directed to cross each other without allowing the droplets to touch, for instance. using a system akin to “traffic lights” to organize flow. For instance, as is shown in FIG. 20, a first stream of droplets 10 and a second stream of droplets 11 are crossed without allowing the droplets to come into direct physical contact. In FIG. 20A., a first stream of droplets 10 (travelling horizontally on the page) is not moved while a second stream of droplets 11 (travelling vertically) is moved. After a certain amount of time, the second stream of droplets 11. is stopped, and the first stream of droplets 10 is then moved. It should be noted that the crossing of two streams of fluidic droplets, in most microfluidic systems, requires the use of a third dimension (e.g., a bypass channel or a “bridge”) in order to prevent the fluidic droplets from contacting each other. Thus, in some embodiments, “channel” geometries or patterns of droplet movements can be implemented that would be difficult or impossible to implement using standard microfluidics. As non-limiting examples, fluidic streams of droplets can cross without requiring actual physical contact, many fluidic streams of droplets may lead to a central point with no outlet (e.g., to coalesce dispersed cells or drops of fluid), or a region may spontaneously spread into a plurality of fluidic streams of droplets.

In another aspect, a fluidic droplet may be split into two or more droplets using electric and/or magnetic fields. The two or more droplets created by splitting the original fluidic droplet may each be substantially the same shape and/or size, or the two or more droplets may have different shapes and/or sizes, depending on the conditions used to split the original fluidic droplet. In many cases, the conditions used to split the original fluidic droplet can be controlled in some fashion, for example, manually or automatically. In some cases, each droplet in a plurality or series of fluidic droplets may be independently controlled. For example, some droplets may be split into equal parts or unequal parts, while other droplets are not split.

In one set of embodiments, a first portion of a fluidic droplet may be urged to move in a first direction, while a second portion of the fluidic droplet may be urged to move into a second direction. The fluidic droplet, in response, may be split into two fluidic droplets. In addition, depending on the electric and/or magnetic fields used to move the fluidic droplet in to the first and second directions, the two “daughter” fluidic droplets may have the same or different sizes.

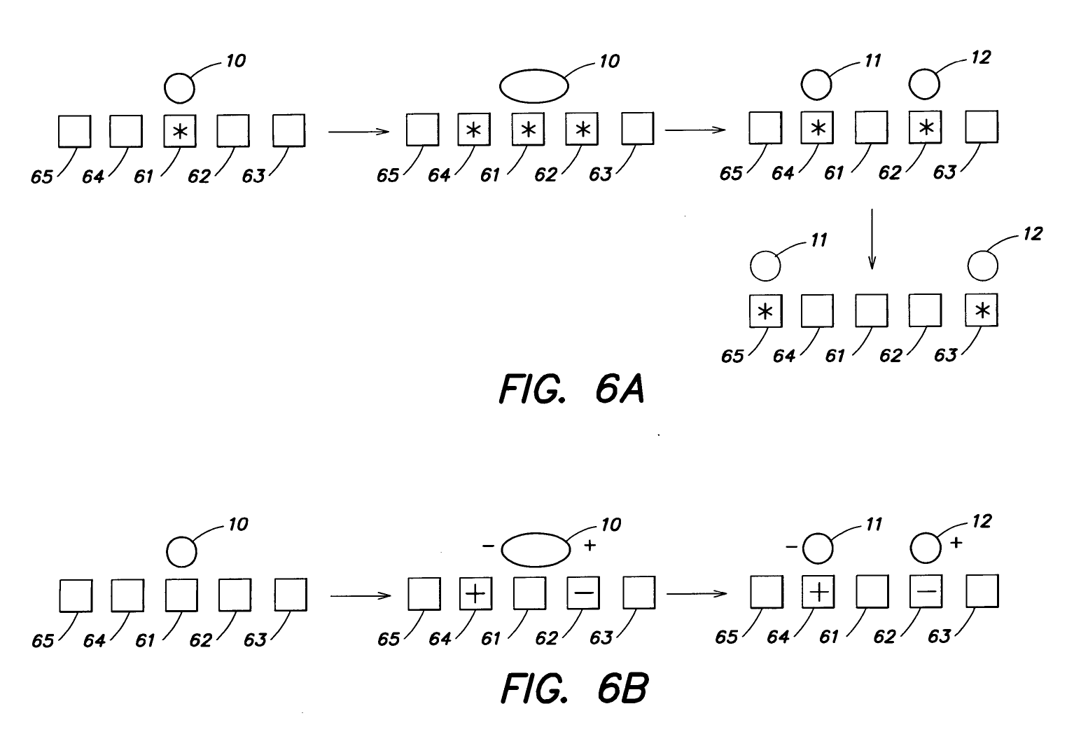

As an example, referring now to FIG. 6A, fluidic droplet 10 is split into daughter fluidic droplets 11 and 12 through activation of field pixels 61-65. In this figure, fluidic droplet 10, proximate field pixel 61, is simultaneously urged to move to the left via the activation of field pixel 62, as previously described, and also urged to move to the right via activation of field pixel 64. The result is that fluidic droplet 10 is split to form daughter droplets 11 and 12, positioned on field pixels 64 and 62, respectively. Subsequently, each individual droplet may then be manipulated using any suitable technique. For instance, FIG. 6A also shows daughter droplets 11 and 12 being moved to field pixels 65 and 63, respectively.

In some cases, the fluidic droplet may be split into three, four, or even more fluidic droplets. For instance, a first portion of a fluidic droplet may be urged to move in a first direction, a second portion of the fluidic droplet may be urged to move into a second direction, and a third portion of a fluidic droplet may be urged to move into a third direction, which may cause the fluidic droplet to become divided into three “daughter” fluidic droplets.

In another set of embodiments, a fluidic droplet can be split using applied electric fields having opposing polarities. The fluidic droplet, in this embodiment, may have a greater electrical conductivity than the surrounding fluid, and, in some cases, the fluidic droplet may be neutrally charged. In certain embodiments, in an applied electric field, electric charge may be urged to migrate from the interior of the fluidic droplet to the surface to be distributed thereon, which may thereby cancel the electric field experienced in the interior of the droplet. In some embodiments, the electric charge on the surface of the fluidic droplet may also experience a force due to the applied electric field, which causes charges having opposite polarities to migrate in opposite directions. The charge migration may, in some cases, cause the drop to be pulled apart into two separate fluidic droplets. As an illustrative example, as is shown in FIG. 6B, fluidic droplet 10, located proximate field pixel 61, is subjected to electric fields of opposite polarity via field pixels 64 and 62. The electric fields may induce charge separation within fluidic droplet 10. As charges within fluidic droplet 10 having opposite polarities migrate in opposite directions, fluidic droplet 10 maybe pulled apart to form two separate fluidic droplets 11, 12.

The invention, in yet another aspect, is directed to fusing or coalescing two or more fluidic droplets into one droplet. For example, in one set of embodiments, systems and methods are provided that are able to cause two or more droplets to fuse or coalesce into one droplet. In some cases, the two or more droplets may fuse or coalesce in cases where the droplets ordinarily are unable to fuse or coalesce, for example, due to composition, surface tension, droplet size, the presence or absence of surfactants, etc. In certain microfluidic systems, the surface tension of the droplets, relative to the size of the droplets, may also prevent fusion or coalescence of the droplets from occurring in some cases.

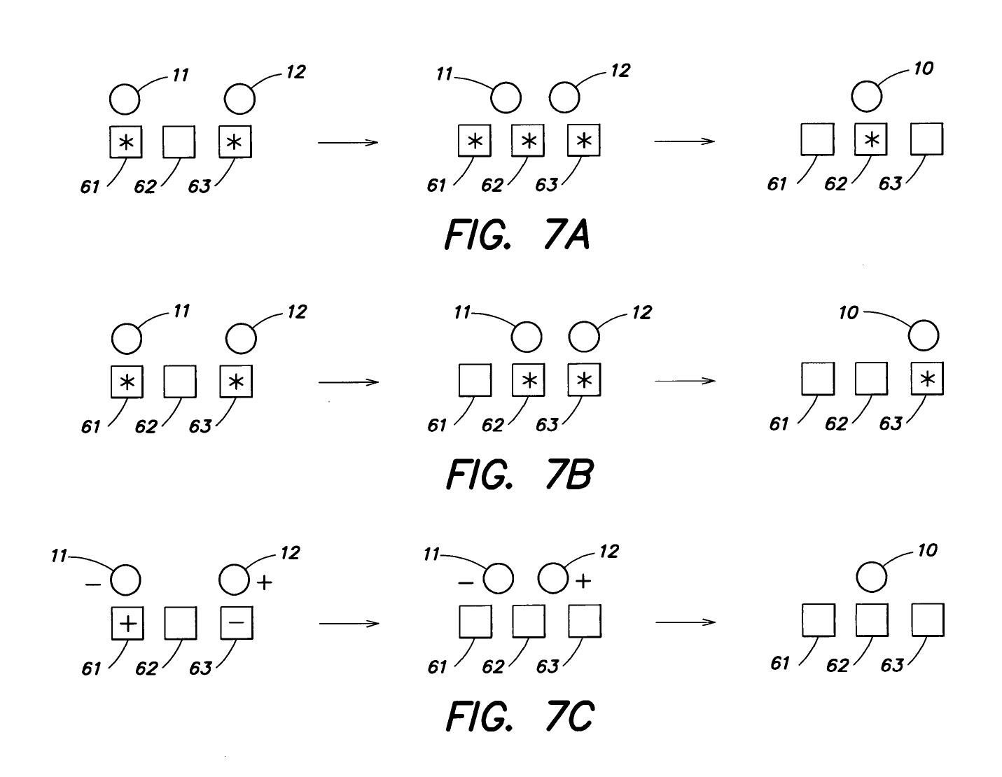

In one set of embodiments, two fluidic droplets may be moved such that the two fluidic droplets come into physical contact with each other (i.e., one or both of the fluidic droplets may be moved such that the droplets come into contact). In some cases, the fluidic droplets may spontaneously coalesce to form a single droplet; however, in other cases, as described below, the droplets may not spontaneously coalesce. For instance, referring now to FIGS. 7A-7B, first fluidic droplet 11 and second fluidic droplet 12 are moved using electric and/or magnetic fields created by through activation of field pixels 61-65, using techniques such as those described above, such that the droplets contact each other to form fluidic droplet 10. In FIG. 7A, both fluidic droplets are moved, while in FIG. 7B, only one fluidic droplet is moved. Of course, the invention is not limited to contacting only two fluidic droplets, and in other embodiments of the invention, three, four, or more fluidic droplets may be urged to come into physical contact with each other.

In another set of embodiments, two fluidic droplets may be given opposite electric charges (i.e., positive and negative charges, not necessarily of the same magnitude), which may increase the electrical interaction of the two droplets such that fusion or coalescence of the droplets can occur due to their opposite electric charges, e.g., using the techniques described herein. For instance, an electric field may be applied to the droplets using one or more electric field-generating components. The droplets, in some cases, may not be able to fuse even if a surfactant is applied to lower the surface tension of the droplets. However, if the fluidic droplets are electrically charged with opposite charges (which can be, but are not necessarily of, the same magnitude), the droplets may be able to fuse or coalesce. For example, referring now to FIG. 7C, fluidic droplets 11 and 12 are given opposite induced electric charges via electric field pixels 61 and 63. Due to their opposite charges, the fluidic droplets are attracted towards each other and coalesce to form fluidic droplet 10, positioned proximate field pixel 62. In some cases, such fluidic droplets may not be able to coalesce in the absence of the induced electric charges.

In a related aspect, the invention allows, in some embodiments, mixing of more than one fluid to occur within a fluidic droplet. For example, in various embodiments of the invention, two or more fluidic droplets may be allowed to fuse or coalesce, as described above, and then, within the fused droplet, the two or more fluids from the two or more original fluidic droplets may then be allowed to mix. In some cases, two or more species may be brought together within the coalesced fluidic droplet to initiate a chemical or a biological reaction, etc. It should be noted that when two droplets fuse or coalesce, perfect mixing within the droplet does not necessarily instantaneously occur.

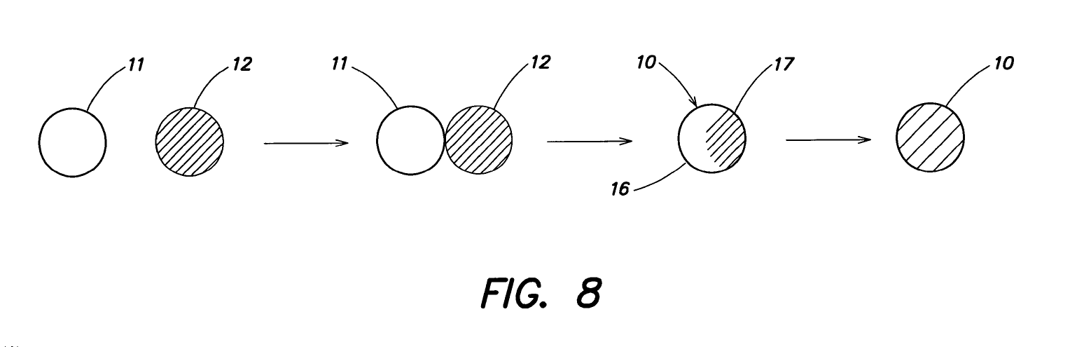

As an example, as is shown in FIG. 8, a coalesced droplet 10 may initially be formed of a first region of fluid 16 (from droplet 11) and a second region of fluid 17 (from droplet 12). The fluid regions can then mix, react, or otherwise interact, eventually forming a coalesced droplet 10 that is partially or completely (i.e., homogeneously) mixed. Mixing of the regions of fluid within the coalesced droplet may be allowed to occur through any suitable mechanism, for example unassisted or natural methods, such as through diffusion (e.g., through the interface between the two regions of fluid), through reaction of the fluids with each other, and/or through fluid flow within the droplet (i.e., convection). In some embodiments, only a portion or a component of a region of fluid (for example, a reactant, as further described below) interacts with other regions of fluid (or a portion or a component thereof), e.g., through mixing, reaction, etc.

In one set of embodiments, the droplets being fused or coalesced may contain reactants (e.g., chemicals, biological molecules, biological entities such as cells, viruses, bacteria, etc.) able to react or otherwise interact with each other. The reactant may be the fluid comprising the droplet and/or a fluidic region within the droplet, and/or the reactant may be carried (e.g., dissolved, suspended, etc.) by a fluid within the droplet and/or within a fluidic region of the droplet. The reaction may be, for example, a precipitation reaction, i.e., the reactants may react in some fashion to produce a solid particle. The reactants may also include, as further non-limiting examples, reactive chemicals, proteins, enzymes/substrates, nucleic acids, proteins/nucleic acids, enzymes/nucleic acids, acids/bases, antibodies/antigens, ligands/receptors, chemicals/ catalysts, etc, as well as combinations of these and other reactants. As another example, one or both droplets may be or contain one or more cells. As yet another example, one droplet that is or contains a cell may be fused with another droplet to create a cell encapsulated in a fluid. Additionally, the fluid may be solidified in some cases to create a cell encapsulated in a solid. As still another example, one droplet may be (or contain) a cell and the other droplet may contain an agent to be delivered to the cell, such as a chemical, a biological molecule, a biological entity, etc., for instance, by fusing a droplet containing the agent with the cell. Non-limiting examples include a nucleic acid (e.g., DNA or RNA, for example, for gene therapy), a protein, a hormone, a virus, a vitamin, an antioxidant, etc. The reaction may be monitored, for example, using sensing moieties such as those described below, using sensors contained within the substrate (e.g., associated with each field generating component), or the like.

In still another aspect, the invention is directed to screening or sorting fluidic droplets in a liquid. For example, a characteristic of a fluidic droplet may be sensed and/or determined in some fashion, for example, as described herein (e.g., fluorescence of the fluidic droplet may be determined), and, in response, the fluidic droplet may be manipulated in some fashion, e.g., moving the fluidic droplet to a particular region (e.g., a channel), splitting the droplet, combining the droplet with another fluidic droplet, or the like.

In certain embodiments of the invention, one or more sensors are provided that can sense and/or determine one or more characteristics of the fluidic droplets, and/or a characteristic of a portion of the fluidic system containing the fluidic droplet (e.g., a liquid surrounding the fluidic droplet) in such a manner as to allow the determination of one or more characteristics of the fluidic droplets. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), or the like. Other non-limiting examples of such sensors include electrical characteristics or magnetic characteristics.

Non-limiting examples of sensors useful in the invention include optical or electromagnetically-based systems. For example, the sensor may be a fluorescence sensor, a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the fluidic system containing the fluidic droplet.

Any suitable property may be sensed or otherwise determined. The property may be a physical property, such as size, density, color (e.g., fluorescence or opacity), temperature, etc., and/or a chemical or a biological property. For instance, the fluidic droplet may contain a sensing entity which can be determined in some fashion, e.g., optically or spectrally. A sensing entity may be one that can interact with another entity such as an analyte (e.g., a chemical, biochemical, and/or biological species) in such a manner to cause a determinable change in a property of the sensing entity. As an example, a sensing entity may fluoresce if a certain analyte is present within the fluidic droplet. For instance, the sensing entity may comprise a binding partner to which the analyte binds. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc. The sensing entity, when it comprises a binding partner, can comprise a specific binding partner of an analyte. For example, the binding partner entity may be a nucleic acid, an antibody, a sugar, a carbohydrate, a protein, an enzyme, etc. Accordingly, by determining the sensing entity within a fluidic droplet, the fluidic droplet may be screened or sorted.

The term “determining,” as used herein, generally refers to the analysis or measurement of a species, for example, quantitatively or qualitatively, and/or the detection of the presence or absence of the species. “Determining” may also refer to the analysis or measurement of an interaction between two or more species, for example, quantitatively or qualitatively, or by detecting the presence or absence of the interaction. Examples of suitable techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements.

Examples of potentially suitable sensing moieties include, but are not limited to, dyes, or fluorescent or chromogenic molecules, for instance, pH-sensitive dyes such as phenol red, bromothymol blue, chlorophenol red, fluorescein, HPTS, 5(6)-carboxy-2′,7′-dimethoxyfluorescein SNARF, and phenothalein; dyes sensitive to calcium such as Fura-2 and Indo-1; dyes sensitive to chloride such as 6-methoxy-N-(3-sulfopropyl)-quinolinim and lucigenin; dyes sensitive to nitric oxide such as 4-amino-5-methylamino-2′,7′-difluorofluorescein; or dyes sensitive to oxygen such as tris(4,4′-diphenyl-2,2′-bipyridine) ruthenium (II) chloride pentahydrate.

In some cases, the sensor may be connected to a processor, which in turn, may cause an operation to be performed on the fluidic droplet, for example, by sorting the droplet, fusing the droplet with another droplet, splitting the droplet, causing mixing to occur within the droplet, etc., for instance, as previously described. For instance, in response to a sensor measurement of a fluidic droplet, a processor may cause the fluidic droplet to be split, merged with a second fluidic droplet, etc. A non-limiting example of a processor is processor 600 in FIG. 1, which may also be connected to various components to facilitate manipulation of the droplet, as discussed herein.

One or more sensors and/or processors may be positioned to be in sensing communication with the fluidic droplet. “Sensing communication,” as used herein, means that the sensor may be positioned anywhere such that the fluidic droplet within the fluidic system may be sensed and/or determined in some fashion. For example, the sensor may be in sensing communication with the fluidic droplet and/or the portion of the fluidic system containing the fluidic droplet fluidly, optically or visually, thermally, pneumatically, electronically, or the like. The one or more sensors can be positioned proximate the fluidic system, for example, embedded within the substrate, associated with one or more field-generating components (e.g., in an array), or positioned separately from the fluidic system but with physical, electrical, and/or optical communication with the fluidic system so as to be able to sense and/or determine the fluidic droplet and/or a portion of the fluidic system containing the fluidic droplet. In some cases, one or more of the field-generating components themselves may also act as sensors.

As an example, a sensor may be free of any physical connection with the fluidic system containing the droplet, but may be positioned so as to detect electromagnetic radiation arising from the droplet or the fluidic system, such as infrared, ultraviolet, or visible light. The electromagnetic radiation may be produced by the droplet, and/or may arise from other portions of the fluidic system (or externally of the fluidic system) and interact with the fluidic droplet and/or the portion of the fluidic system containing the fluidic droplet in such as a manner as to indicate one or more characteristics of the fluidic droplet, for example, through absorption, reflection, diffraction, refraction, fluorescence, phosphorescence, changes in polarity, phase changes, changes with respect to time, etc. “Sensing communication,” as used herein may also be direct or indirect. As an example, light from the fluidic droplet may be directed to a sensor, or directed first through a fiber optic system, a waveguide, etc., before being directed to a sensor.

As a non-limiting example, in a sample containing a plurality of droplets of fluid, some of which contain a species of interest and some of which do not contain the species of interest, the droplets of fluid may be screened or sorted for those droplets of fluid containing the species (e.g., using fluorescence or other techniques such as those described above), and in some cases, the droplets may be screened or sorted for those droplets of fluid containing a particular number or range of entities of the species of interest, e.g., as previously described. Thus, in some cases, a plurality or series of fluidic droplets, some of which contain the species and some of which do not, may be enriched (or depleted) in the ratio of droplets that do contain the species, for example, by a factor of at least about 2, at least about 3, at least about 5, at least about 10, at least about 15, at least about 20, at least about 50, at least about 100, at least about 125, at least about 150, at least about 200, at least about 250, at least about 500, at least about 750, at least about 1000, at least about 2000, or at least about 5000 or more in some cases. In certain embodiments, the droplets carrying the species may then be fused, reacted, or otherwise used or processed, etc., as further described below, for example, to initiate or determine a reaction.

As another non-limiting example, a device of the invention may contain fluidic droplets containing one or more cells. The cells may be exposed to a fluorescent signal marker that binds if a certain condition is present, for example, the marker may bind to a first cell type but not a second cell type, the marker may bind to an expressed protein, the marker may indicate viability of the cell (i.e., if the cell is alive or dead), the marker may be indicative of the state of development or differentiation of the cell, etc., and the cells may be directed through a fluidic system of the invention based on the presence/absence, and/or magnitude of the fluorescent signal marker. For instance, determination of the fluorescent signal marker may cause the cells to be directed to one region of the device (e.g., a collection chamber), while the absence of the fluorescent signal marker may cause the cells to be directed to another region of the device (e.g., to be directed to a waste chamber). Thus, in this example, a population of cells may be screened and/or sorted on the basis of one or more determinable or targetable characteristics of the cells, for example, to select live cells, cells expressing a certain protein, a certain cell type, etc.

In one set of embodiments, control of fluids within the channels may be included in a feedback system, where the droplets are moved dynamically in response to sensor or other information regarding the fluidic droplets. One example of such a feedback system is described in Example 1. In some cases, the fluidic droplets may be determined using an optical microscope, for example, connected to a camera such as a digital camera. Objects such as cells or droplets of fluids can be sorted with such a system, for instance, based on measurements of the droplet's optical properties, properties of species within the droplets, or the like.

In some cases, droplets or cells may be moved along various pixels in a predetermined manner or in in a series of pre-programmed patterns. For example, the pixels may be pre-programmed to be able to manipulate droplets, for example, by moving, sorting, splitting, coalescing, reacting, etc., the droplets. As a specific non-limiting example, a property of a first droplet may be determined (e.g., fluorescence), and the droplet then directed, using a pre-programmed pattern of pixels, to a first location or to a second location. In some cases, the use of pre-programmed patterns may allow for faster or easier to implement sorting modality, for example, by limiting the number of locations that a droplet may be found at. The pre-programmed patterns, for instance, may be used to bring cells or fluid droplets into a region where they are inspected and then sorted.

As mentioned, certain aspects of the invention include fluidic systems comprising one or more microfluidic components, for example, one or more microfluidic channels. “Microfluidic,” as used herein, refers to a fluidic system that includes at least dimension of less than about 1 mm. For example, as illustrated in FIG. 1, fluidic system 300 may include a chamber 301 having at least one dimension that is less than about 1 mm, as well as microfluidic channels 302 and 304 to facilitate fluid flow into and out of chamber 301. A “microfluidic channel,” as used herein, is a channel meeting these criteria. The “cross-sectional dimension” of the channel is measured perpendicular to the direction of fluid flow within the channel. Thus, some or all of the fluid channels in microfluidic embodiments of the invention may have maximum cross-sectional dimensions less than 2 mm, and in certain cases, less than 1 mm. In one set of embodiments, all fluid channels containing embodiments of the invention are microfluidic or have a largest cross sectional dimension of no more than 2 mm or 1 mm. In certain embodiments, the fluid channels may be formed in part by a single component (e.g. an etched substrate or molded unit). Of course, larger channels, tubes, chambers, reservoirs, etc. can also be used, e.g., to store fluids, manipulate fluids, and/or to deliver fluids to various components or systems of the invention. In one set of embodiments, the maximum cross-sectional dimension of the channel(s) containing embodiments of the invention is less than 500 microns, less than 200 microns, less than 100 microns, less than 50 microns, or less than 25 microns.

A “channel,” as used herein, means a feature on that at least partially directs flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and/or outlet(s). A channel may also have an aspect ratio (length to average cross sectional dimension) of at least 2:1, more typically at least 3:1, 5:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

As a specific, non-limiting example, as illustrated in FIG. 1, fluidic system 300 may be a microfluidic system that includes an essentially rectangular-shaped chamber 301 above an IC chip 102 that contains a two-dimensional array of field-generating components 200 disposed in a plane proximate to and essentially parallel to a floor of the chamber. Such an arrangement may facilitate manipulation of samples, such as fluidic droplets generally along two dimensions defining a plane parallel to the floor of the chamber (indicated by x-y axes in FIG. 1), e.g., as previously discussed. In another implementation, field-generating components may alternatively or additionally be disposed along one or more sides of such a chamber to facilitate manipulation of samples or droplets along a third dimension transverse (e.g., perpendicular) to the floor of the chamber (indicated by a z axis in FIG. 1). In yet another implementation, a chamber may be “sandwiched” between two arrays of field-generating components respectively contained in IC chips disposed above and below the chamber. In such an arrangement, the multiple arrays of field-generating components may be controlled such that three-dimensional manipulation of samples or droplets may be accomplished. Additionally, various arrangements of field-generating components with respect to the microfluidic system may facilitate rotation of samples.

It should be appreciated that the foregoing exemplary arrangements are provided primarily for purposes of illustration, and that a variety of arrangements of a fluidic system and field-generating components (including linear or two-dimensional arrays of field-generating components, or other arrangements of discrete field generating components) are contemplated according to other embodiments to provide multi-dimensional manipulation of samples. In general, according to the various concepts discussed herein, samples of interest, such as fluidic droplets, may be moved through the fluidic system along virtually any path, trapped or held at a particular location, and in some cases rotated, under computer control of the electric and/or magnetic fields generated by the field-generating components. In this manner, the topology of a “virtual micro-scale plumbing system” for samples of interest may be flexibly changed for a wide variety of operations based on the programmability and computer control afforded, for example, by one or more processors.

A variety of materials and methods, according to certain aspects of the invention, can be used to form the fluidic or microfluidic system. For example, various components of the invention can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al).

In one set of embodiments, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE” or Teflon®), or the like. For instance, according to one embodiment, system 100 shown in FIG. 1 may be implemented by fabricating fluidic system 300 separately using PDMS and soft lithography techniques, and subsequently attaching the microfluidic system to the IC chip 102 (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In some embodiments, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Another advantage to forming microfluidic structures of the invention (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

In one embodiment, a bottom wall is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, thermal bonding, solvent bonding, ultrasonic welding, etc.

In another set of embodiments, at least a portion of system 100 may include components fabricated using CMOS technologies. For instance, IC chip 102 may be fabricated using CMOS technologies, using techniques known to those of ordinary skill in the art. In some cases, a layer of a CMOS chip may include a silicon nitride or polyimide passivation layer, whose purpose is to prevent chemical elements such as sodium from penetrating into the chip. A fluidic system, such as a microfluidic system, may be further fabricated on the top of the CMOS chip passivation layer in certain embodiments of the invention. In some cases, the fluidic system may include micropatterned polyimide sidewalls in desired shapes so as to form channels, chambers, reservoirs, or the like. For example, once diced, portions of substrate 104 may be spin-coated with polyimide and then patterned using conventional lithography techniques. Since the CMOS chip surface layer generally includes a polyimide passivation layer, micropatterned polyimide sidewalls can be fabricated with good adhesion to the similar-material passivation layer. In various exemplary implementations, the coating and patterning process for the polyimide layer may be configured to form a height and width for a fluidic channel or a microfluidic channel, depending on the requirements of a given application.

In some cases, after the fabrication of a fluidic channel in a polyimide layer, according to one embodiment, the surface of the fluidic channel may be optionally coated (e.g., spin-coated) with a thin layer of polydimethylsiloxane, or PDMS. PDMS is a biocompatible material whose surface can be functionalized to either encourage or prevent cell adhesion. For example, in one aspect of this embodiment, treating the oxidized surface of polymerized PDMS with fibronectin (FN) makes it amenable to micro-patterning of extracellular matrix proteins to facilitate cell adhesion and spreading. In another aspect, treating the surface of PDMS with Pluronic F127 can block protein absorption, thus preventing the adhesion of cells. These respective characteristics may facilitate different aspects of guiding biological samples down the microfluidic channels of a cell sorter according to one embodiment of the present disclosure, and for directing the cells to specific locations during two-dimensional micro-scale tissue assembly according to another embodiment of the present disclosure, as previously discussed. In various implementations, PDMS may be spin-coated to micron-thickness layers onto the surface of the fluidic channel, without compromising sample manipulation or imaging.

In some instances, a cover slip (e.g., a glass cover slip) may be coupled to a polyimide layer, e.g., forming a microfluidic chamber or channel. The surface of the cover slip to be joined to the polyimide layer may be coated with a negative photoresist or ultraviolet curable epoxy (e.g., SU-8, available from Microchem, Inc. of Newton, Mass.) to facilitate a seal between the cover slip and the polyimide layer (e.g., via curing of the assembly with ultraviolet light). In one implementation, a UV curable photoresist or epoxy again may be used to bond the tube fittings and conduits to the assembly.

The following are incorporated herein by reference: U.S. patent application Ser. No. 10/894,674, filed Jul. 19, 2004, entitled “Methods and Apparatus Based on Coplanar Striplines,” by Ham, et al., published as U.S. Patent Application Publication No. 2005/0068116 on Mar. 31, 2006, now U.S. Pat. No. 7,091,802, issued Aug. 15, 2006; U.S. patent application Ser. No. 10/894,717, filed Jul. 19, 2004, entitled “Methods and Apparatus Based on Coplanar Striplines,” by Ham, et al., published as U.S. Patent Application Publication No. 2005/0068127 on Mar. 31, 2005; International Patent Application No. PCT/US02/36280, filed Nov. 5, 2002, entitled “System and Method for Capturing and Positioning Particles,” by Westervelt, et al., published as WO 03/039753 on May 15, 2003; and U.S. patent application Ser. No. 11/105,322, filed Apr. 13, 2005, entitled “Methods and Apparatus for Manipulation and/or Detection of Biological Samples and Other Objects,” by Ham, et al., published as U.S. Patent Application Publication No. 2006/0020371 on Jan. 26, 2006.

In addition, the following are also incorporated herein by reference: U.S. patent application Ser. No. 11/024,228, filed Dec. 28, 2004, entitled “Method and Apparatus for Fluid Dispersion,” by Stone, et al., published as U.S. Patent Application Publication No. 2005/0172476 on Aug. 11, 2005; U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/360,845, filed Feb. 23, 2006, entitled “Electronic Control of Fluidic Species,” by Link, et al., published as U.S. Patent Application Publication No. 2007/0003442 on Jan. 4, 2007; and International Patent Application No. PCT/US2006/007772, filed Mar. 3, 2006, entitled “Method and Apparatus for Forming Multiple Emulsions,” by Weitz, et al., published as WO 2006/096571 on Sep. 14, 2006. Also incorporated herein by reference is U.S. Provisional Patent Application Ser. No. 60/947,063, filed Jun. 29, 2007, entitled “Methods and Apparatus for Manipulation of Fluidic Species,” by Hunt, et al.

The following examples are intended to illustrate certain embodiments of the present invention, but do not exemplify the full scope of the invention.

### Example 1

This example, and the following examples, illustrate the development and testing of a hybrid IC/microfluidic system. The device in this example was able to trap and move many small volumes of fluid or biological cells independently in a system that could be dynamically programmed and could receive and react to feedback signals. The chip in this example moved cells and fluid drops using dielectrophoresis (DEP), the motion of dielectrics in non-uniform electric fields.

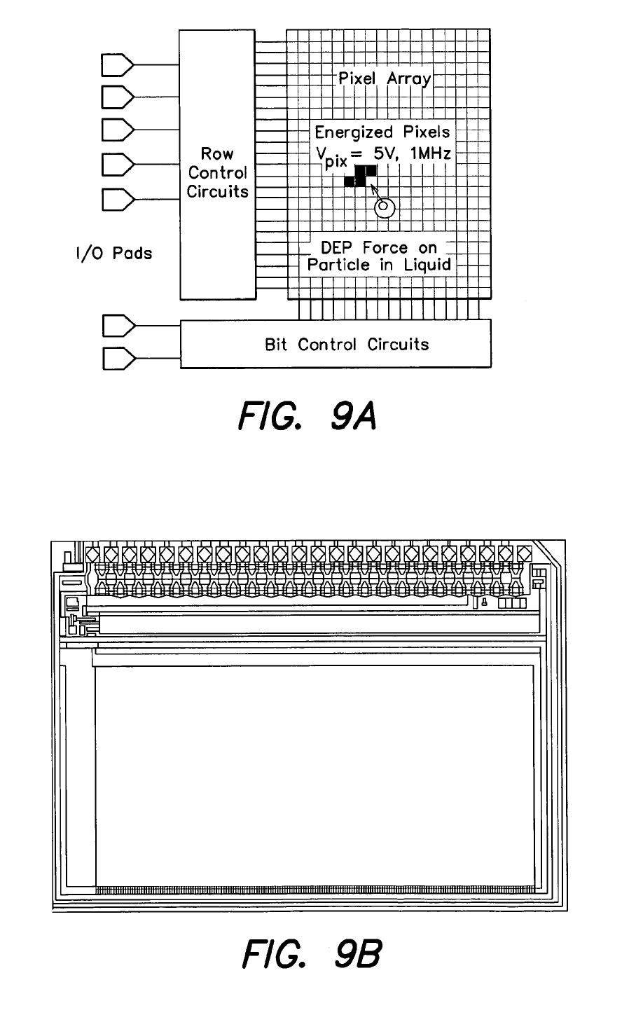

This example illustrates a hybrid IC/microfluidic system that included a microfluidic structure built on top of a custom IC manufactured in a foundry. The IC had an array of pixels similar in architecture to a computer display or a digital camera (FIG. 9). In this device, each pixel was individually driven with a radio frequency (RF) voltage, creating a local electric field that exerted a force on cells or drops of fluid above the chip's surface in the microfluidic chamber via DEP. With this pixel geometry, programmed micro-patterned, time-dependent RF electric fields could be formed and could be used to trap and move objects in fluid above the chip.

The IC demonstrated here was built using a standard complementary metal on oxide process (CMOS) design and lithographic process. The chip was formed from a 1.4×2.8 mm² array of 32,768 individually addressable 11×11 micrometer² pixels. An RF voltage with an amplitude of 5 V at frequencies from DC to 1.8 MHz could be applied to each pixel producing a localized electric field to trap a cell or drop of fluid. Subsequently, a microfluidic chamber was fabricated on its top surface.

This example also demonstrates how the hybrid chip could be programmed to trap and move individual yeast and mammalian cells in solution. Also, thousands of individual yeast cells could be trapped and simultaneously positioned into controlled patterns. In addition, this example shows the chip translating, splitting, and mixing water droplets in oil.

The chip in this example included an array of 128×256 pixels and was surrounded by control circuitry to address and control the pixels, as is show in FIG. 9A. The row control circuits selected a row of pixels and the bit control circuits selected a column, allowing individual pixels to be addressed. Each pixel contained a memory element that stores its state. A micrograph of the integrated circuit is shown in FIG. 9B.

A 0.35-micrometer gate length CMOS process with four metal layers and 5 V transistors, available through MUSTS (process: TSMC35_P2), was selected. The process was chosen to provide strong field gradients for DEP and a pixel size to match cellular size scales (i.e., on the order of 10 micrometers). A summary of the IC design parameters is shown in Table 1.

TABLE 1

| | |
|---|---|
| Process | 0.35 micrometer, CMOS MOSIS TSMC |
| Pixels | 128 × 256 (32,768), 11 × 11 micrometer² pixels |
| Chip Size | 2.32 × 3.27 mm² |
| Addressing | 8-bit word line decoder; 128-bit, two-phase |
| | clocked shift register for bit lines |
| Transistor Count | ~360,000 |
| Pixel Voltage | vₚᵢₓ = 3 to 5 V, DC to 1.8 MHz |
| Operating Current | 30 to 100 mA |

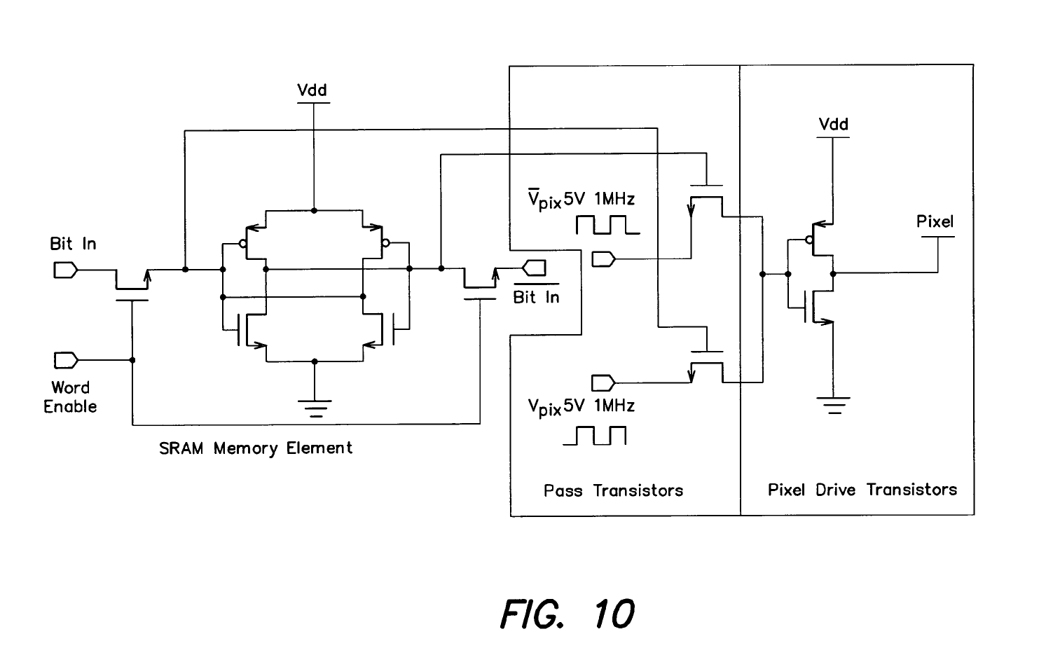

The circuit diagram of a single pixel in the chip is shown in FIG. 10. Each pixel included three basic circuit blocks: a static random access memory (SRAM) element to store the state of the pixel; control transistors that, depending on the state of the SRAM, allows either vₚᵢₓ or the logical inverse v̅ₚᵢₓ to be applied to the DEP electrode; and drive transistors to pull-up and pull-down the capacitive load of the pixel.

The RF voltage vₚᵢₓ and its inverse v̅ₚᵢₓ were signals that are created off of the chip. vₚᵢₓ is an RF square-wave with a 50% duty cycle and a frequency that can range from DC to 1.8 MHz. The memory element of each pixel determined whether the pixel is driven with vₚᵢₓ or v̅ₚᵢₓ . There were several advantages to driving pixels with either vₚᵢₓ or v̅ₚᵢₓ rather than vₚᵢₓ or a DC value. The electric field between pixels held at vₚᵢₓ and v̅ₚᵢₓ time averaged to zero, so there was no electrophoresis of charged particles in the microfluidic system. Also, the RMS electric field attainable between vₚᵢₓ and v̅ₚᵢₓ was twice the RMS electric field that was attainable between vₚᵢₓ and ground, providing a greater DEP force than having just vₚᵢₓ relative to ground. In addition, the time that it took for the pixel voltage to ramp up or down was short compared to the period of vₚᵢₓ. The transistors that drove each pixel had an on-resistance of approximately 10 kilohms (kΩ) and drove a pixel capacitance less than 50 fF, yielding a sub-nanosecond RC time.

To maximize the pixel density of the IC, the number of transistors under each pixel was minimized and the circuit layout was optimized to pack transistors as densely as possible. To further conserve chip area, all PMOS transistors for pixels on a common word line shared an N-doping well. To facilitate the fabrication of the microfluidic system, all bond pads were located on one side of the chip. Input/output (IO) pads were designed to provide 1.6 kV ESD protection without consuming excessive chip area.

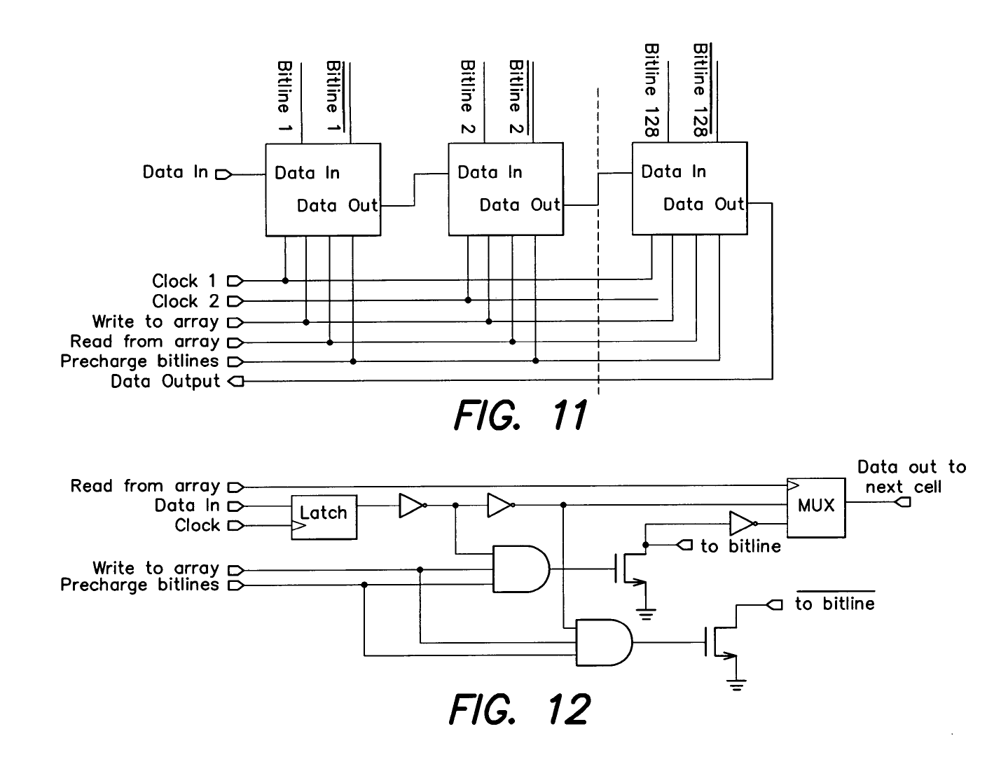

To address the 128×256 pixels in the array, various circuits were used to selectively update the states of the pixels by their individual rows and columns. To select one of the 256 rows, a decoder was used that identified each row with a unique 8 digit binary number. The state of each of the 128 pixels in a row were loaded sequentially into a shift register, as shown in FIG. 11, and then simultaneously written to the designated row. In FIG. 11, the dashed line represents blocks that serve bit line 3 to bit line 127.

The shift register was updated using a 2 phase clocking scheme, with Clock1 and Clock2. Control signals Read and Write determined whether the bits in a row were written from the shift register or were read. The memory states were written using a 2 phase clocked pre-charged logic. The schematic of each element of the shift register is shown in FIG. 12. To set the pixel values of one word of pixels on the chip, data for each pixel was loaded into a two-phase clocked shift register. Bitline precharging was disabled, the write to array signal was given, and bitlines corresponding to data in each latch were pulled down by NMOS transistors. An 8-bit word line decoder enabled one of the 256 word lines on the chip to be written, and the bitline values were written to the SRAM elements on the selected word.

To non-destructively read the SRAM memory elements on the chip and confirm which pixels are energized, bitline precharging was disabled, a wordline was enabled, and all bits of the selected word were read to the 128 latches. Subsequent two phase clocking stepped the latch values through the final latch to an output amplifier (digital inverter, output current of 10 microamperes (μA), and on to an output pin.

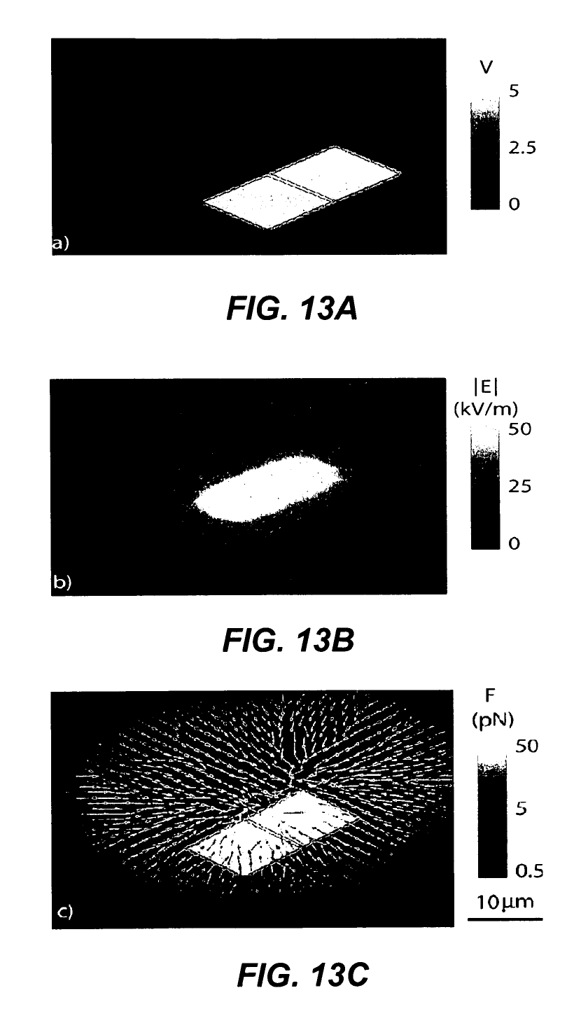

FIG. 13 shows simulated electric and force fields for an 8 micrometer diameter sphere above the chip with the dielectric properties of a cell in a water bath. The simulation geometry was modeled on the actual chip geometry: 10.4×10.4 micrometer metal pixels, with a 0.6 micrometer spacing in either direction, capped with 3 micrometers of polyimide and 200 micrometers of water above the surface of the chip. In the simulation, 2 pixels were set to 5 V, leaving all other pixels at ground (FIG. 13A). Finite element simulations were used to determine the electric field 4 micrometers above the surface of the chip (FIG. 13B), from which x,y components of the DEP force acting on the center of an 8 micrometer diameter cell in the microfluidic channel (FIG. 13C). The simulations were executed with Maxwell 3D (Ansoft Inc.).

The simulations showed that a cell in the microfluidic channel was exposed to a maximum electric field of about 50 kV/m, and that an 8 micrometer diameter cell above one electrode would be subject to a DEP force of approximately 5 pN when a neighboring electrode was energized.

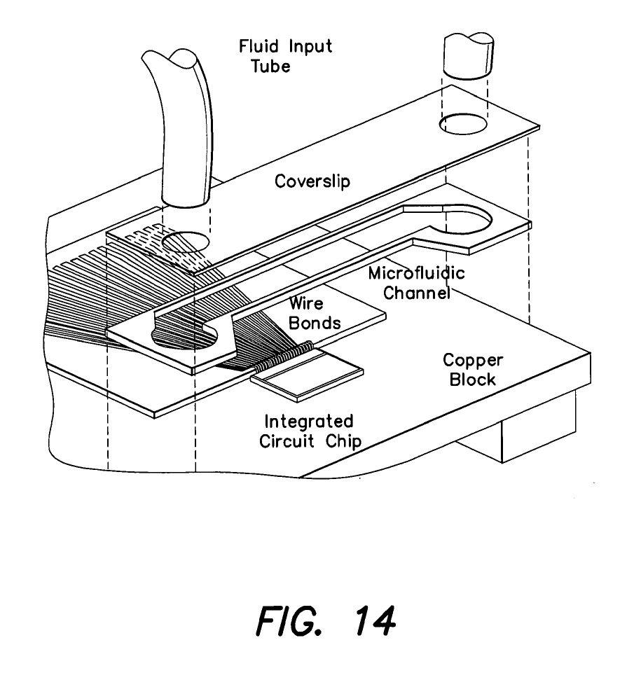

The microfluidic packaging scheme is shown in FIG. 14. An IC was first mounted on a copper block, for heat transfer, and then the IC was wirebonded to microfabricated leads placed next to the IC. The microfluidic channels were formed using hot-melt adhesive for the channel walls. A cover slip with drilled fluid ports was placed onto and thermally adhered to the channel walls. With hot-melt channel walls, the cover slip could be removed to clean the surface of the chip or the entire channel could be replaced by moderately heating the chip and peeling back the thermally bonded layer. The IC dies were received from a foundry and all subsequent processing was done in a lab.

To fabricate a microfluidic channel for mounting on top of the IC, a sheet of hot melt adhesive was cast. The fluid channel walls were formed by setting adhesive between spaced silanized glass slides on a hotplate at 100° C. The thin layer of adhesive was peeled off and the microfluidic channel was cut with a hole punch designed for microfluidics (Harris Uni-core, Pella Inc.). Under a binocular dissecting microscope, the microfluidic channel was aligned onto the IC surface while heating the chip to approximately 90° C.

Two schemes were used to introduce fluid into the fluidic channel. A few microliters of liquid could be injected with a pipette to directly fill the microfluidic channel and a coverslip placed on top. Alternatively, a coverslip with drilled via holes could be thermally bonded to the hot melt channel. With the thermally bonded coverslip, fluid could be injected with syringe pumps into the microfluidic channel through the holes. However, it should be noted that any standard microfluidic system could constructed on the top side of the coverslip to supply reagents, cells, fluidic drops, or the like to the chip.

To control and program the hybrid chip, the IC was electrically connected to a computer controller. The interface between the IC and the computer was a printed circuit board (PCB). Control signals were sent to the PCB by a National Instruments PCI-6254 board mounted in a personal computer. The RF voltage, vₚᵢₓ, was provided by a function generator, and v̅ₚᵢₓ by an inverter on the PCB. The computer ran a custom user interface written in Igor Pro (Wavemetrics, Inc.), with NI-DAQ software to control the NI board. The PCB also regulated the power lines and protected inputs to the IC with RC filters. The PCB was designed with PCAD (Altium, Ltd.), in the Harvard Electronics Shop, and was manufactured by Advanced Circuits.

The IC in this particular example was designed for a 1 MHz pixel read and write rate; however, the NI board had a limited update rate of approximately 20 kHz. This I/O speed allowed a word of 128 pixels to be updated at ˜100 Hz which was adequate for these experiments.

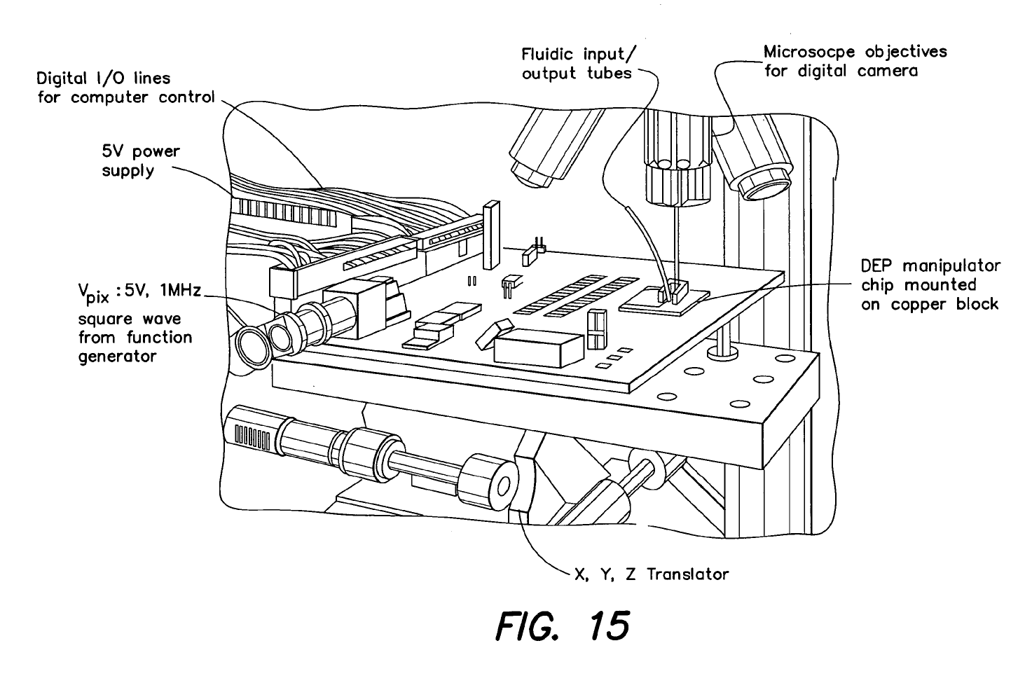

FIG. 15 shows the IC/microfluidic DEP manipulator chip experimental setup described in this example. The PCB containing the hybrid chip was mounted on a microscope stage. Wirebonds connect the electrical leads to the circuit board and were protected from fluid and mechanical damage by a layer of PDMS.

The chip was constructed with a 0.35 micrometer, 5 V CMOS process as a compromise between expense, pixel size and actuation voltage. A 0.35 micrometer gate width is several generations behind current CMOS technology, and more narrow gate widths can thus be used in other embodiments. For instance, Intel has demonstrated a 0.57 micrometer² SRAM in their 65 nm production process. A DEP chip with an identical architecture to this chip requires only four transistors per pixel in addition to the basic SRAM building block. As a result of the progress of the semiconductor industry, it is straightforward to design DEP pixels 1×1 micrometer² instead of 11×11 micrometer², given the teachings herein.

In semiconductor scaling, smaller transistors may have lower breakdown voltage. The semiconductor industry has purposefully pursued lower voltage to maximize switching speed while minimizing power dissipation, using 0.9-1.2 V power supplies for the 65 nm processes. With slight modifications, specifically a thicker gate oxide, switching speed can be slowed for increased gate-source voltage. The source-drain breakdown voltage could also be increased by adding a lightly doped drift region to each transistor.

Even without process modification, a DEP chip fabricated with a 65 nm, 1 V CMOS process produces strong electric fields and field gradients for DEP due to the short separation between pixels. The passivation thickness above the metal layers may be scaled with the pixel size, or the field gradient used for DEP will fall off within the passivation. Small DEP manipulator chips constructed with semiconductor technology could also be used for positioning nanoparticles in complex patterns. Post-processing with nano-lithography, such as electron beam lithography, could be useful in applying this approach to the nanoscale.

### Example 2

This example demonstrates how the hybrid chip of Example 1 could be programmed to trap and move individual yeast cells in solution.

Yeast cells were cultured overnight in YPD broth (BD Inc.) at 37° C. The conductivity of the broth was approximately 1 S/m as measured by an Orion 116 conductivity meter (Thermoelectron Inc.). The yeast were resuspended in a mannitol buffer, with a conductivity of 100 microsiemens/m to reduce the effects of heating and electrohydrodynamic flow in the strong electric fields produced by the DEP chip. Approximately 5 microliters of yeast cells in mannitol were pipetted onto the chip.

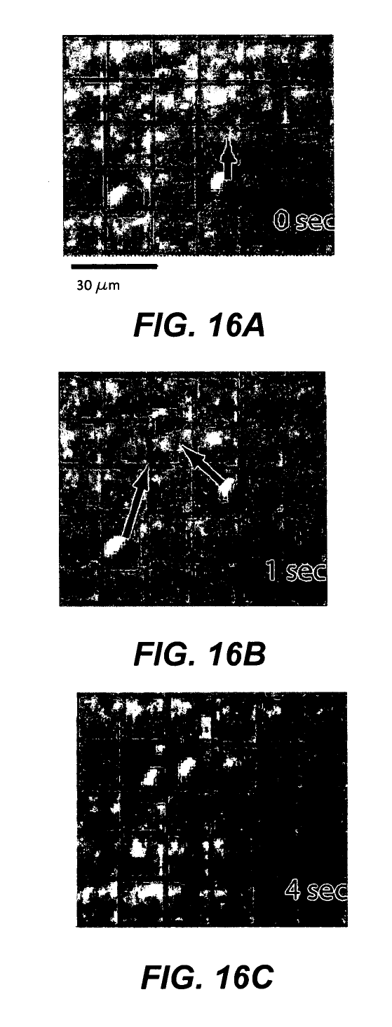

FIG. 16 shows microscope images of yeast cells trapped and moved by the chip. At 0 seconds, a few pixels were energized with vₚᵢₓ, while all of the other pixels were driven with v̅ₚᵢₓ. Three yeast cells were captured in the maximum of the electric field above the energized pixels (FIG. 16A). By changing which pixels were energized, individual cells were moved from one pixel to a neighboring pixel at approximately 30 micrometers/s. After 1 second (FIG. 16B), two of the yeast cells were moved to their final position at 4 seconds (FIG. 16C). It was possible to move any cell along an arbitrary path by energizing a sequence of electrodes. It was also possible to separate two neighboring cells by rapidly switching the pattern of energized pixels.

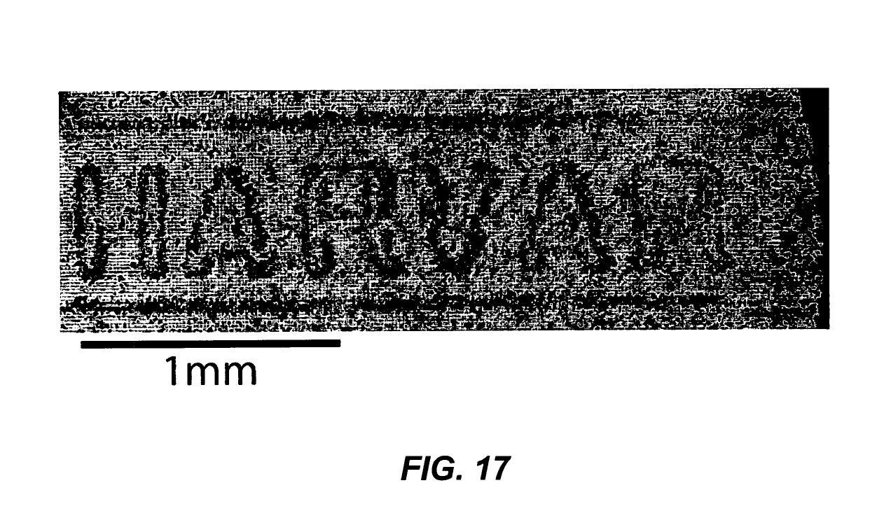

By appropriately addressing the pixel array, thousands or more individual cells could be simultaneously trapped and moved in any arbitrary fashion. For instance, FIG. 17 shows yeast cells that have been moved to form a programmed pattern with the DEP array. Pixels were energized in a bitmap that spelled “Harvard” and yeast cells in mannitol were pipetted onto the chip surface. As the cells sedimented, they were attracted to the local maxima in the electric field produced by the pattern of energized electrodes on the chip surface. The image was taken once the cells had settled to the surface of the chip, ˜10 minutes after introducing the yeast suspension. This sort of directed cell positioning thus has application in tissue assembly applications.

### Example 3

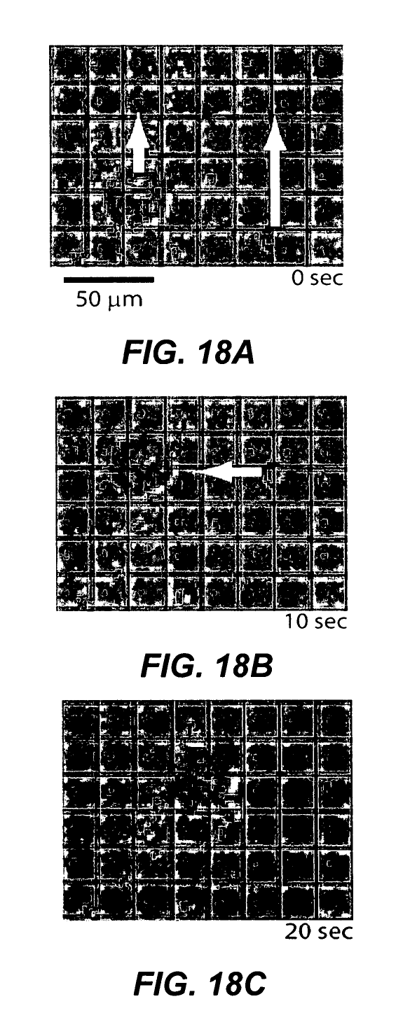

In addition to yeast cells, mammalian cells could also be manipulated, as is shown in FIG. 18. In this example, rat alveolar macrophages were trapped and moved in the same manner as the yeast cells described above. To demonstrate the potential of the chip described in Example 1 to assemble tissue from multiple cell types and to enable studies of cell-cell interaction, both rat alveolar macrophages and yeast cells were simultaneously moved here. In this example, multiple yeast cells were delivered to the surface of a rat alveolar macrophage, with control of the distance between cells of different types.

Rat alveolar macrophages were prepared in the Bioimaging Lab at Harvard School of Public Health. The cells were obtained by bronchoalveolar lavage and suspended in a low conductivity buffer, 0.1 M sucrose to avoid heating and EHD flow. Residual ions brought the conductivity of the sucrose buffer to 100 microsiemens/M.

### Example 4

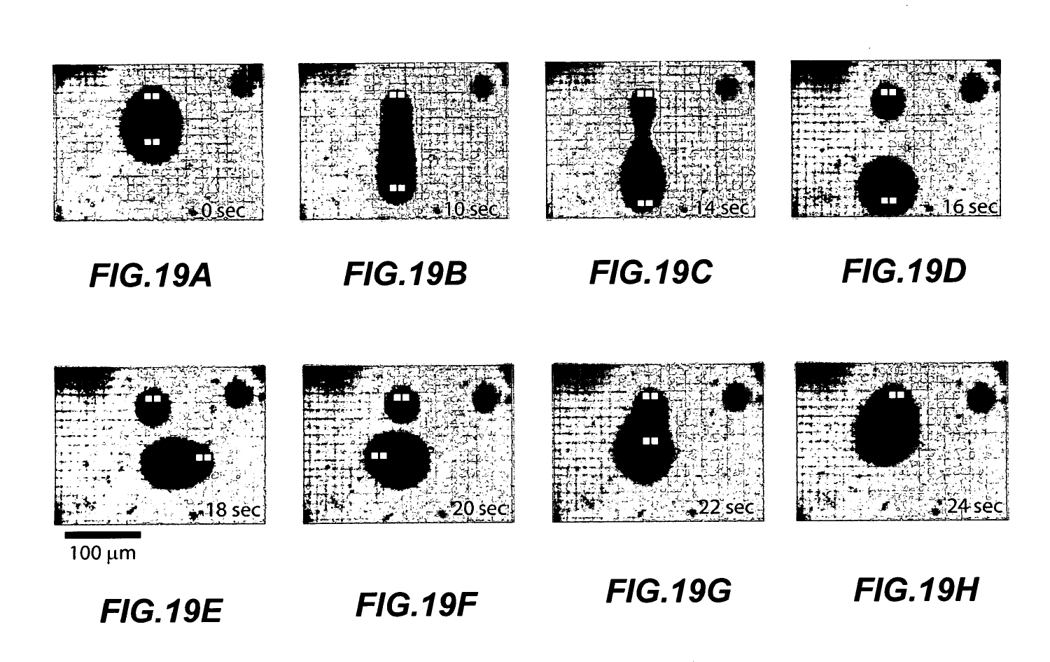

This example illustrates use of the DEP manipulator chip described in Example 1 to move, split, or combine drops of water in oil. Water drops with volumes from ˜1 nL to ˜1 pL were programmably manipulated by the electric fields produced by the chip. FIG. 19 shows the DEP manipulation of dyed water drops in oil with energized pixels highlighted in white. The time is shown in the lower left corner of each figure. Droplets were deformed by energizing multiple sets of pixels. While holding a droplet in place with two energized pixels, another set of pixels was energized to stretch the droplet (FIGS. 19A-19C). As the drop is stretched, the single droplet was pinched off into two separate droplets due to surface tension (FIG. 19D). The two droplets were then moved independently (FIGS. 19E-19F). The droplets were then recombined when they were brought into contact (FIG. 19G-19H).

To prepare drops for manipulation, a mixture of hexadecane, water, and sodium dodecyl sulphate (SDS) surfactant was shaken using a vortexer. A thin layer of fluorocarbon oil was pipetted onto the surface of the chip and then the suspension of water drops in hexadecane was added to the microfluidic channel. The difference in density among the three liquids resulted in water drops (eta, η=1 gm/cm³) that were pinched between a layer of dense fluorocarbon oil (eta, η=2.4 gm/cm³) and less dense hexadecane (eta, η=0.8 gm/cm³) The multilayer liquid provided very little resistance to translating drops in 2D above the surface of the chip. The droplets were not in contact with the chip surface so it was not necessary to overcome contact line hysteresis to move the drops. In addition, droplet manipulation was insensitive to the surface treatment and hydrophobicity of the chip.

Integrated circuit/microfluidic systems capable of droplet manipulation could serve as a platform for programmable, automated chemistry. Reservoirs of chemicals along the edge of the chip could be used to deliver fluid droplets, pinched off with DEP, and mixed together in any programmable pattern to perform a wide variety of biochemical assays. In addition, programmable control of droplets allows pL chemical doses to be delivered directly to drops that hold cells. The chip also allows deforming a droplet and mixing the contents of a droplet faster than simple diffusive mixing.

### Example 5

Without wishing to be bound by any theory, it is believed that certain aspects of dielectrophoresis theory are applicable to certain embodiments of the invention, as discussed in this example.

In general, DEP is the motion of a dielectric in a non-uniform electric field. A non-uniform electric field creates an induced electric dipole in a dielectric. An induced dipole moment feels a force in the non-uniform field. By applying an appropriate local electric field, any particle with a dielectric constant different than the surrounding medium can be manipulated with DEP. The DEP force on a spherical particle is:

F̅_{DEP}(ω)=²πεₘα³CM(ω)∇Eᵣₘₛ² (1),

where α is the radius of the particle, εₘ (epsilon m) is the medium permittivity, and CM(ω) (omega) is the Clausius-Mossotti factor, a relation between the frequency dependent complex permittivity of the particle and the medium, and εₚ is the complex permittivity of the particle:

$$CM ( ω ) = Re [ \frac{{\hat{ɛ}}_{p} - {\hat{ɛ}}_{m}}{{\hat{ɛ}}_{p} + 2 {\hat{ɛ}}_{m}} ] . \qquad ( 2 )$$

CM(ω) (omega) can vary between −0.5 and 1 with important physical implications. When CM(ω) (omega) is less than 0, the fluid is more polarizable than the particle and the particle is pulled toward the local minimum of the electric field, this is called negative DEP (nDEP). Positive DEP (pDEP) occurs when the particle is more polarizable than the fluid, i.e., CM(ω) (omega) is greater than 0, and the particle is pulled to the maximum of the electric field.

The hybrid chip in the above examples uses pDEP to move cells and droplets in the experiments described. By shifting the location of energized pixels, the array changes the location of the local electric field maxima, trapping and moving cells along programmable paths through the microfluidic chamber. In both model and experiment, a conductive coverslip was unnecessary for cell and droplet manipulation. This system was equally capable of nDEP manipulation, with confinement in the Z-direction provided by gravity or a coverslip.

There are several reasons to use AC fields in this DEP. AC fields allow ion shielding of the electrodes to be avoided. In a conductive medium, AC fields of sufficient frequency (>10 kHz) do not suffer from ionic screening or electrode polarization: ions cannot move fast enough to screen the applied field. The movement of particles due to net charge (electrophoresis) will time average to zero in an AC field and electroosmotic flow of the double layer along liquid-solid boundaries is eliminated. Another benefit of AC fields is that they are less harmful to cells, because the voltage across the capacitive membrane of the cell is less than that with a DC field.

It can be calculated that a cell in the above-described DEP chip experiences a maximum transmembrane voltage of ˜30 mV due to the applied electric field of 50 kV/m at 1 MHz. A number of studies have shown that cells subject to less than 10⁵ V/m at frequencies greater than 1 MHz show few signs of damage due to the applied electric field.

Control of droplets of aqueous chemicals in oil is a major accomplishment for this IC/microfluidic system. These examples demonstrate that IC/microfluidic systems can serve as a platform for programmable, automated chemistry. Reservoirs of chemicals along the edge of the chip can be used to deliver fluid droplets, pinched off by DEP, and/or mixed together in any programmable pattern to perform a wide variety of biochemical assays. In addition, programmable control of droplets allows picoliter (pL) quantities of chemical doses to be delivered directly to droplets that contain single cells.

While several embodiments of the present invention have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one,

A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-6. (canceled)

7. A method, comprising:
manipulating a fluidic droplet, separated from a substrate by a fluid layer substantially immiscible with the fluidic droplet, using at least one electric and/or magnetic field generated from an array of field-generating components contained within the substrate.

8. The method of claim 7, wherein the fluidic droplet has a characteristic dimension of less than about 1 mm.

9. The method of claim 7, wherein the fluidic droplet contains a cell.

10. The method of claim 7, wherein the array of field-generating components are CMOS fabricated.

11-36. (canceled)

37. A method, comprising:
generating one or more electric and/or magnetic fields by activating one or more field-generating components of a plurality of field-generating components contained within a substrate; and
manipulating a fluidic droplet not in direct contact with the substrate using the one or more electric and/or magnetic fields.

38. The method of claim 37, wherein the sample is a fluidic droplet.

39-51. (canceled)

52. The method of claim 38, wherein the fluidic droplet is separated from the substrate by a separating fluid.

53-54. (canceled)

55. The method of claim 52, further comprising a covering fluid covering at least a portion of the separating fluid, wherein the fluidic droplet is positioned between the covering fluid and the separating fluid.

56-96. (canceled)

97. An apparatus, comprising:
a plurality of CMOS fabricated field-generating components;
a microfluidic system containing fluid in proximity to the plurality of CMOS fabricated field-generating components, the fluid comprising a first fluid layer, a second fluid layer, and a fluidic droplet contained between the first fluid layer and the second fluid layer; and
at least one controller configured to control the plurality of CMOS fabricated field-generating components to generate at least one electric or magnetic field having a sufficient strength to interact with at least one sample suspended in the fluid.

98. (canceled)

99. The apparatus of claim 97, wherein the at least one controller is configured to control the plurality of CMOS fabricated field-generating components to generate a plurality of programmable spatially or temporally variable electric or magnetic fields having a sufficient strength to interact with the at least one sample suspended in the fluid.

100. The apparatus of claim 99, further comprising at least one processor coupled to the at least one controller, the at least one processor configured to control the at least one controller so as to facilitate at least one of manipulation, detection, imaging and characterization of the at least one sample via the plurality of electric or magnetic fields.

101. (canceled)

102. The apparatus of claim 97, wherein the at least one controller includes a plurality of CMOS fabricated field control components forming an integrated circuit chip together with the plurality of CMOS fabricated field-generating components.

103-106. (canceled)

107. The apparatus of claim 102, wherein the plurality of field control components includes:
a plurality of programmable switching or multiplexing components; and
a plurality of current or voltage sources.

108. The apparatus of claim 107, wherein the plurality of field control components further includes a plurality of high frequency detection components configured to facilitate at least one of detection, imaging and characterization of the at least one sample suspended in the fluid via the generated at least one electric or magnetic field.

109-111. (canceled)

112. The apparatus of claim 97, wherein the plurality of CMOS fabricated field-generating components includes a plurality of microcoils.

113. (canceled)

114. The apparatus of claim 112, wherein each microcoil includes at least two axially concentric spatially separated portions of conductor turns.

115. The apparatus of claim 112, wherein the at least one controller includes a plurality of switching or multiplexing components and a plurality of current or voltage sources coupled to the plurality of microcoils.

116. The apparatus of claim 115, wherein the at least one controller further includes a plurality of radio frequency (RF) detection components coupled to the plurality of microcoils.

117. The apparatus of claim 116, wherein the plurality of RF detection components includes a frequency locked loop configured to facilitate at least one of detection, imaging and characterization of the at least one sample suspended in the fluid.

118. The apparatus of claim 117, wherein the frequency locked loop includes at least one bridge circuit, the at least one bridge circuit including at least one microcoil of the plurality of microcoils, the at least one bridge circuit configured to generate at least one signal representing a change in an inductance of the at least one microcoil due to a presence of the at least one sample in proximity to the at least one microcoil.

119-124. (canceled)
